(19) United States
(12) Patent Application Publication
(10) Pub. No.: US 2003/0129168 A1
(43) Pub. Date: Jul. 10, 2003

(54) Expression of proteins for treating asthma via ligand mediated activation of their encoding genes

(75) Inventor: Franklin Cerasoli JR., Marlborough, MA (US)

Correspondence Address: ARIAD Gene Therapeutics, Inc., 26 Landsdowne Street, Cambridge, MA, 02139, US

(73) Assignee: ARIAD Gene Therapeutics, Inc.

(21) Appl. No.: 10/087,286

(22) Filed: Mar. 1, 2002

Related U.S. Application Data
(63) Continuation of application No. 09/367,170, filed on Aug. 9, 1999, now abandoned.
(60) Provisional application No. 60/032,260, filed on Dec. 9, 1996.

(30) Foreign Application Priority Data
Dec. 9, 1997 (US) PCT/US97/22454

(51) Int. Cl. A61K 48/00; C12N 5/08; C12N 15/86

(52) U.S. Cl. 424/93.21; 435/455; 435/456; 435/320.1; 435/366

(57) ABSTRACT

A method for treating or preventing asthma in a mammal involving administering to the mammal an effective dose of a drug capable of activating expression of a recombinant DNA molecule encoding a target protein in genetically engineered cells present within the mammal.

### FIELD OF THE INVENTION

This invention concerns regulated gene therapy and its use for treating or preventing asthma and related disorders.

### BACKGROUND OF THE INVENTION

Asthma has been defined as “a lung disease having the following characteristics: (1) airway obstruction that is reversible (but not completely so in some patients) either spontaneously or with treatment; (2) airway inflammation; and (3) increased airway responsiveness to a variety of stimuli.” See “Guidelines for the Diagnosis and Management of Asthma,” J. Allergy Clin. Immunol. 88:425-534 (1991). Asthma also has been defined as “a chronic inflammatory disorder of the airwavs in which many cells play a role, including mast cells and eosinophils. In susceptible individuals, this inflammation causes symptoms which are usually associated with widespread but variable airflow obstruction that is often reversible either spontaneously or with treatment, and causes an associated increase in airway responsiveness to a variety of stimuli.” See Clin. Exper. Allergy 80:1-72 (1992).

There are currently two major classes of asthma therapies: 1) symptomatic or “rescue” therapy, and 2) preventative or “disease modifying” therapy.

Symptomatic therapies include methyl xanthines, such as theophylline and aminophylline, and -agonists, such as salmeterol, fenoterol and albuterol. The use of theophylline has been and may still be the mainstay of bronchodilatory therapy for asthma in the United States. However, administration of theophylline is complicated by the need to titer patients carefully to ensure that the dose administered to a given patient is efficacious but avoids toxic side effects, including those caused by hemodynamic effects. β-agonists are believed to be the most common bronchodilators at the present time and are available in a variety of strengths and durations of activity, including highly potent, less potent, long acting, and short acting. β-agonists are primarily intended to promote bronchodilation of constricted airways in order to provide symptomatic relief for the asthmatic. β-agonists are sometimes administered prophylactically in anticipation of bronchoconstrictive events, such as exercise, immersion into cold air, immersion into antigenic environments (e.g., pollen clouds) and stressful situations.

With the more recent development of long acting β-agonists, the demarcation between symptomatic relief and preventative therapy has become less dear. β-agonists are typically viewed as having no disease modifying activities. However, long acting β-agonists can be taken before going to bed to prevent the characteristic decline in the early morning peak expire flow rate that many asthmatics experience. Despite such use, the main purpose of β-agonists remains symptomatic relief of acute “attacks”. Typically β-agonists are the agents prescribed to mild asthmatics who experience infrequent asthmatic episodes, although this has been reported to be changing. Thus, β-agonists are a “rescue” therapy for moderate and severe asthmatics and are part of add-on medication regimens.

An ideal form of asthma therapy would be disease-modifying, and would thus limit the need for β-agonists or other bronchodilators as “rescue” therapy. Current disease modifying therapies include steroids, cromolyn, nedocromil sodium, methyl xanthines such as theophylline, and more recently, leukotriene D4 receptor antagonists. These agents are not completely active and thus may not prove sufficiently efficacious in a given patient. For patients that are not sufficiently responsive to initial therapy(ies), several other therapies are often tested at various dosages in an effort to optimize treatment. Those therapies may be tested sequentially or in combination. In the latter case, the patient is typically weaned to the minimum effective dose of the minimum number of drugs.

Steroids are generally considered to be the most efficacious drugs for treating asthma. Steroids decrease airway inflammation, airway hyperreactivity and episodic acute bronchoconstriction. Steroids also permit decreased use of β-agonists. Inhaled steroids are considered to be reasonably safe, but do possess various side effects which can include weight gain, HPA-axis imbalance, retarded growth, and sexual malfunction. Oral steroids can be provided to severe asthmatics, but can cause significant side effects, generally more severe than in the case of inhaled steroids. Some patients (“steroid resistant” asthmatics) fail to respond adequately to steroids.

One aim of newer (“steroid-sparing”) therapies is to avoid the need to administer corticosteroids to patients. Therapies based on LTD₄ active agents, new and more potent phosphodiesterase inhibitors, thromboxane receptor antagonists, immunotherapy, methotrexate, cyclosporin and PAF receptor antagonists are all designed to be -anti-inflammatory, decrease acute bronchoconstriction, eliminate airway hyperreactivity, be steroid sparing and reduce the need for rescue therapy with β-agonists.

At present, preferred asthma therapies involve administration of the drug by nebulizer or metered dose inhaler (MDI). However, the efficacy of such administration can be compromised by inconsistent or inadequate use of devices for the respective therapies. This often occurs with the elderly and children. Patient compliance can be problematic as a result of the “ernbarrassment factor” of carrying a device on one's person and using it in public.

The present invention provides a new approach for achieving the therapeutic goals mentioned above and does so with methods and materials intended to avoid. some or all of the side effects and other shortcomings of current therapies.

### SUMMARY OF THE INVENTION

This invention provides a method and materials for treating or preventing asthma in a mammal, preferably a human subject, which contains cells genetically engineered to permit the regulated expression of one or more of IL-10, IL-12, gamma interferon or a nitric oxide (“NO”) synthase. The invention involves the administration to the mammal of a therapeutically effective dose of a ligand which is capable of activating expression of at least one of the foregoing target proteins in genetically engineered cells within the mammal, preferably cells within the mammal's airways. The gamma interferon, interleukin 10 (IL-10), interleukin 12 (IL-12) and NO synthase (“NOS”) products, as those terms are used herein, denote full-length proteins of naturally occurring human peptide sequence, as well as peptides, fragments, subunits and analogs of the foregoing which retain one or more of the characteristic biological activities of the parent protein, e.g. inhibition of TH2 cell function or other anti-inflammatory activity. When this invention is applied to human patients, it is preferred that the target protein comprise a naturally occurring human peptide sequence. In the case of NOS, it should be appreciated that a number of species with NOS activity are known and may be adapted for use in this invention, including the following:

| | Genbank | | | |
|---|---|---|---|---|
| | Accession | | | |
| Protein | # | Source | Author | Reference |
| Human | U20141 | airway | Guo et al. | PNAS USA 92(17) |
| iNOS | | epithelium | | 7809-7813, 1995 |
| Human | L09210 | hepatocytes | Geller et al. | PNAS USA 90(8) |
| iNOS | | | | 3491-3495, 1993 |
| Human | U05810 | chondrocytes | Maier et al. | BBA 1208(1) |
| iNOS | | | | 145-150, 1994 |
| Human | D26525 | glioblastoma | Hokari et al. | J. Biochem 116(3), |
| iNOS | | cell line A-172 | | 575-581, 1994 |
| Human | M95296 | vascular | Marsden et | FEBS Lett. 309, |
| ceNOS | | endothelium | al. | 287-293, 1992 |
| Human | U17327 | brain | Hall et al. | JBC 269(52), |
| nNOS | | | | 33082-33090, 1994 |

To render the mammal responsive to the ligand, certain of the mammal's cells must first be genetically engineered by introducing into them heterologous DNA constructs, typically in vivo. A variety of systems have been developed which permit the genetic engineering of cells to permit ligand-mediated regulatable expression of a target gene (although heretofore, none have been applied to the treatment or prevention of asthma as provided herein). See e.g. Clackson, “Controlling mammalian gene expression with small molecules”, Current Opinion in Chemical Biology 1:210-218 (1997). Materials and methods for implementing those systems are known in the art and may be adapted to the practice of the subject invention. Typically, at least two different heterologous DNA constructs are introduced into the cells, including (a) at least one target DNA construct which comprises a target gene, here a DNA sequence encoding a target protein, i.e., IL-10, IL-12, IFN-gamma or a nitric oxide synthase operably linked to a transcription control element permitting ligand-mediated expression of the target gene; and (b) one or more DNA constructs encoding and capable of directing the expression of chimeric proteins capable of binding to the ligand and activating expression of the target gene(s) in a ligand-dependent manner.

Preferred regulated expression systems are based on ligand-mediated dimerization of chimeric proteins. In such systems each of the chimeric proteins contains at least one ligand-binding (i.e., receptor) domain and at least one effector domain for activating gene transcription directly or indirectly. The phrase “ligand-binding domain” encompasses protein domains which are capable of binding to the ligand, as in the case of an FKBP domain and the ligand, FK506, discussed below, and further encompasses protein domains which are capable of binding to a complex of the ligand with another binding protein, as in the case of the FRB domain which binds to the rapamycin:FKBP complex. Examples of pairs of receptor domains and ligands which are known in the art and have been demonstrated to be effective in such regulated transcription systems, and which may be used in the practice of the subject invention, include FKBP/FK1012, FKBP/synthetic divalent FKBP ligands (see WO 96/0609 and WO 97/31898), FRB/rapamycin:FKBP (see e.g., WO 96/41865 and Rivera et al, “A humanized system for pharmacolgic control of gene expression”, Nature Medicine 2(9):1028-1032 (1997)), cydophilin/cyclosporin (see e.g. WO 94/18317), DHFR/methotrexate (see e.g. Licitra et al, 1996, Proc. Natl. Acad. Sci. USA 93:12817-12821) and DNA gyrase/coumermycin (see e.g. Farrar et al, 1996, Nature 383:178-181).

In the case of direct activation of transcription, two chimeric proteins are typically used. Each, as mentioned above, contains at least one ligand-binding domain. One of the chimeras also contains at least one DNA-binding domain such as GAL4 or ZFHD1; the other contains at least one transcription activation domain such as VP16 or the p65 domain from NF-kappaB. The presence of a ligand to which the two chimeric proteins can bind, and through which the chimeric proteins can complex with one another to form protein dimers or multimers, activates transcription of a target gene linked to a transcription control element containing a DNA sequence which is recognized by, i.e., binds to, the DNA-binding domain. Typically the transcription control element also includes a minimal promoter sequence. DNA binding domains and transcription activation domains for use in treating human subjects preferably comprise human peptide sequence, as represented by ZFHD1 and p65. The transcription control element of a target gene construct to be directly activated by ligand-mediated dimerization will typically contain multiple copies of a recognition sequence for the DNA-binding domain and a minimal promoter.

In the case of systems for the indirect activation of transcription, at least one of the chimeric proteins also contains at least one ligand-binding domain and at least one effector domain. However, in these embodiments the effector domain comprises a cellular signaling domain such as the cytoplasmic domain of a growth factor receptor, which upon association with one or more like domains triggers transcription of a gene linked to a responsive promoter. Said differently, mutual association of such effector domains is considered to transmit an intracellular signal, which results in the activation of a responsive promoter. For example, clustering of the cytoplasmic portion of the zeta chain of the T Cell receptor triggers transcription of a gene linked to an IL-2 promoter. Numerous promoters responsive to the mutual association of various signaling domains are well known. See e.g. pages 23-26 of PCT/US94/01617 (WO 94/18317). The foregoing may be adapted to the subject invention to provide effector domains for the chimeric proteins and responsive promoters for target DNA constructs.

Alternatively, there are several ligand-mediated regulated transcription systems which are based on mechanisms other than ligand-mediated dimerization which, while not preferred, may be adapted to the practice of the subject invention. In these systems, binding of ligand to a chimeric protein activates transcription of a target gene linked to a responsive transcription control sequence.

One such sytem relies upon a chimeric protein comprising a GAL4 DNA binding domain, a ligand-binding domain derived from the human progesterone receptor hPRB891 and the VP16 activation domain. The target gene construct comprises a target gene linked to a transcription control sequence comprising GAL4 binding sites. Administration of the progesterone antagonist RU 486 activates expression of the target gene. See e.g. Wang et al, 1994, Proc. Natl. Acad. Sci. USA 91:8180-8184. If used in the practice of the subject invention, it would be preferred to use DNA binding and activation domains of human origin, such as ZFHD1 and p65, in place of GAL4 and VP16.

Another such system relies upon a chimeric protein comprising a DNA binding domain and a ligand-binding domain derived from an ecdysone receptor VpEcR or VgEcR. The target gene construct comprises a target gene linked to a transcription control sequence comprising an ecdysone-responsive promoter. Administration of ecdysone or muristerone A as the ligands activates expression of the target gene. See e.g. No et al, 1996, Proc. Natl. Acad. Sci. USA 93:3346-3351.

Still another such system relies upon a chimeric protein, rtTA, comprising a modified Tet repressor domain and the VP16 transcription activation domain which in the presence of tetracycline or an analog thereof such as doxycydine activates transcription of a target gene linked to the bacterial tet operon. If used in the practice of the subject invention, it would be preferred to use a transcription activation domain of human origin in place of VP16. See e.g. Gossen et al, 1995, Science 268:1766-1769.

Whichever of the foregoing approaches is elected, the desired DNA constructs are then generally incorporated into a DNA vector and introduced into the cells. Various methods and materials for doing so are known in the art and may be adapted to the practice of this invention, including the introduction of so-called “naked DNA” and the use of viral vectors. Viral vectors useful in gene therapy are well known and include, among others, retroviruses, vaccinia viruses, pox viruses, adenoviruses and adeno-associated viruses (AAV). Any viral vector useful in gene therapy may be used in the practice of this invention.

For in vivo gene therapy in accordance with the subject invention, the viral or other DNA vector containing the desired DNA constructs is administered to the mammal in a sufficient amount to transfect a sufficient number of cells and render them capable of ligand-mediated expression of the target gene to provide a therapeutic benefit upon administration of the ligand. Of the various routes of administration known in the art, any routes permitting transfection of cells of the manunal's airways are preferred (e.g. airway epithelial cells, airway smooth muscle cells, etc.). Such routes include instillation of vector DNA through a bronchoscope and inhalation of vector DNA, e.g. in nebulized or aerosolized form.

Dosages of the DNA will depend on the choice of target gene(s), design of the DNA constructs, potency of ligand-dependent target gene expression, type of vector used, and route of administration, as well as on factors such as the severity of the condition being treated, the age, weight and condition of the mammal. Accepted therapeutically effective dosages of viral vectors for use on human subjects is generally in the range of about 20 to about 50 ml of saline solution containing from about 1×10⁷ to 1×10¹⁰ pfu/ml of viruses. Dosage decisions will typically be made by a patient's physician taking into account the foregoing factors. Effectiveness of the transfection or infection may be determined, if desired, by analysis, using conventional molecular biological techniques, of cells recovered from the recipient via bronchoalveolar lavage or biopsy. If desired, the transfection may be repeated.

Once the mammal has been suitably transfected or infected with the desired DNA constructs, one may administer the ligand to the mammal to treat or prevent the occurrence of asthma. Typically the ligand is administered in the form of a pharmaceutical composition containing the ligand and one or more carriers and optional excipients as described below. Such pharmaceutical compositions may be administered by any of the varied routes of administration used for delivering pharmaceutical agents, although oral, sublingual, bucal and other routes of administration other than injection or inhalation are currently preferred for ligands which are sufficiently bioavailable by those routes. Preferably, the pharmaceutical composition will be taken on a regular schedule, e.g. once or twice per day or per week, rather than upon the occurrence of an asthmatic episode. The precise dosing and scheduling decisions should be made by the attending physician taking into account factors such as those mentioned in the preceding paragraph and elsewhere herein.

### DETAILED DESCRIPTION OF THE INVENTION

The definitions and orienting information below will be helpful for a full understanding of this document.

The terms “protein”, “polypeptide” and “peptide” are used interchangeably herein.

“DNA constructs” as that term is used herein, are generally either target DNA constructs or regulatory DNA constructs. The former are polynucleotides comprising a coding region (“target gene” encoding a “target protein”) operably linked to a transcription control element permitting ligand-dependent regulated expression of the target gene. Regulatory DNA constructs comprise a coding region encoding a chimeric protein which activates transcription of the target gene in the presence of a ligand to which it binds, or in some embodiments, in the presence of a second chimeric protein and a common ligand to which both chimeric proteins bind. That coding region is operably linked to a transcription control element which in many embodiments comprises a strong constitutive promoter such as the hCMV promoter.

The term “transcription control element” denotes a regulatory DNA sequence, such as initiation signals, enhancers, and promoters, which induce or control transcription of protein coding sequences with which they are operably linked. The term “enhancer” is intended to include regulatory elements capable of increasing, stimulating, or enhancing transcription from a promoter. Such transcription regulatory components can be present upstream of a coding region, or in certain cases (e.g. enhancers), in other locations as well, such as in introns, exons, coding regions, and 3′ flanking sequences.

The term “activate” as applied herein to the expression or transcription of a gene denotes a directly or indirectly observable increase in the production of a gene product.

“Recombinant”, “chimeric” and “fusion”, as those terms are used herein, indicate that the various component domains or sequences are mutually heterologous in the sense that they do not occur together in the same arrangement, in nature. More specifically, the component portions are not found in the same continuous polypeptide or nucleotide sequence or molecule in nature, at least not in the same cells or order or orientation or with the same spacing present in the chimeric protein or recombinant DNA molecule of this invention.

“Dimerization”, “oligomerization” and “multimerization” refer to the association of two or more proteins, mediated, in the practice of this invention, by the binding of each such protein to a common ligand. The terms are used interchangeably herein. The formation of a tripartite (or greater) complex comprising proteins containing one or more FKBP domains together with one or more molecules of an FKBP ligand which is at least divalent (e.g. FK1012 or AP1510) is an example of such association or clustering. In cases where at least one of the proteins contains more than one ligand binding domain, e.g., where at least one of the proteins contains three FKBP domains, the presence of a divalent ligand leads to the clustering of more than two protein molecules. Embodiments in which the ligand is more than divalent (e.g. trivalent) in its ability to bind to proteins bearing ligand binding domains also can result in clustering of more than two protein molecules. The formation of a tripartite complex comprising a protein containing at least one FRB domain, a protein containing at least one FKBP domain and a molecule of rapamycin is another example of such protein clustering. In certain embodiments of this invention, fusion proteins contain multiple PRB and/or FKBP domains. Complexes of such proteins may contain more than one molecule of rapamycin or a derivative thereof and more than one copy of one or more of the constituent proteins. Again, such multimeric complexes are still referred to herein as tripartite complexes to indicate the presence of the three types of constituent molecules, even if one or more are represented by multiple copies. The formation of complexes containing at least one divalent ligand and at least two molecules of a protein which contains at least one ligand binding domain may be referred to as “oligomerization” or “multimerization”, or simply as “dimerization”, “clustering” or association”.

“Divalent”, as that term is applied to ligands in this document, denotes a ligand which is at least divalent with respect to proteins containing a receptor domain which binds to the ligand. Said differently, a divalent ligand is capable of complexing with at least two protein molecules which contain ligand binding domains, to form a three (or greater number)-component complex.

“Genetically engineered cells” denotes cells which have been modified by the introduction of recombinant or heterologous nucleic acids (e.g. one or more DNA constructs or their RNA counterparts) and further includes the progeny of such cells which retain part or all of such genetic modification.

A “therapeutically effective dose” of a ligand denotes a treatment or prophylaxis effective dose, e.g., a dose which yields detectable prophylaxis or reduction in the severity of symptoms of asthma, a dose which measurably activates expression of the target gene in the genetically engineered cells as determined by measurement of target protein levels, or a dose which is predicted to be treatment or prophylaxis effective by extrapolation from data obtained in animal or cell culture models.

I. Regulation of Target Gene Transcription

While various approaches to the regulation of transcription are available, as discussed above, dimerization-based approaches to regulated transcription are preferred as is the use of chimeric proteins which contain protein domains of human origin, or derivatives thereof. Currently preferred ligand binding domains are based on FKBP12, and in some cases, the FRB domain of FRAP. Those domains may be engineered to recognize novel FKBP ligands and/or rapamycin derivatives, as disclosed in PCT/US94/01617 and PCT/US96/09948 (WO 96/41865). Preferred DNA-binding domains include ZFHD1 and related composite DNA binding domains as disclosed in PCT/US95/16982 (WO 96/20951) along with the DNA sequences they recognize.

Depending on design preferences of the practitioner, a wide variety of ligands may be used. In general, ligands for use in this invention are preferably non-proteinaceous and preferably have a molecular weight below about 5 kD, more preferably below about 3 kD. FK1012, cyclosporin-based divalent ligands, fujisporin and related types of semisynthetic ligands are disclosed in WO 94/18317 and PCT/US94/08008 (WO 95/02684). Ligands based on synthetic FKBP ligand monomers are disclosed in WO 96/06097 and WO 97/31898, and ligands based on rapamycin and derivatives are disclosed in WO 96/41865. Ligands for the ecdysone receptor, tet system and other proteins are disclosed in various cited references, including those cited and discussed above. All of the foregoing components may be used in the practice of this invention and the full contents of the various documents referred to above are incorporated herein by reference. Those documents also provide guidance in the design of constructs encoding such chimeras, expression vectors containing them, design and use of suitable target gene constructs, and their use in engineering host cells. As further guidance in that regard, specific examples are provided below which illustrate the design, construction and use of constructs for the regulated expression of target genes using direct regulation (DNA binding domains and activation domains) and indirect regulation (dimerization of signal transduction domains).

FKBP, FRB, cyclophilin and other ligand binding domains comprising naturally occurring peptide sequence may be used in the design of chimeric proteins for use in practicing this invention. Alternatively, domains derived from naturally occurring sequences but containing one or more mutations in peptide sequence, generally at up to 10 amino acid positions, and preferably at 1-5 positions, more preferably at 1-3 positions and in some cases at a single amino acid residue, may be used in place of the naturally occurring counterpart sequence and can confer a number of important features. This is described at length in the previously cited patent documents, together with numerous examples of such mutations and corresponding ligands, all of which are incorporated at this point specifically in that regard.

For example, illustrative mutations of current interest in FKBP domains include the following:

| | | | |
|---|---|---|---|
| F36A | Y26V | F46A | W59A |
| F36V | Y26S | F48H | H87W |
| F36M | D37A | F48L | H87R |
| F36S | I90A | F48A | F36V/F99A |
| F99A | I91A | E54A | F36V/F99G |
| F99G | F46H | E54K | F36M/F99A |
| Y26A | F46L | V55A | F36M/F99G |

note: Entries identify the native amino acid by single letter code and sequence position, followed by the replacement amino acid in the mutant. Thus, F36V designates a human FKBP12 sequence in which phenylalanine at position 36 is replaced by valine. F36V/F99A indicates a double mutation in which phenylalanine at positions 36 and 99 are replaced by valine and alanine, respectively.

Illustrative FRB mutations, especially for use with rapamycin analogs bearing substituents other than -OMe at the C7 position include amino acid substitutions for one of more of the residues Tyr2038, Phe2039, Thr2098, Gln2099, Trp2101 and Asp2102. Exemplarv mutations include Y2038H, Y2038L, Y2038V. Y2038A, F2039H, F2039L, F2039A, F2039V, D2102A, T2098A, T2098N, andT2098S. Rapamycin derivatives bearing substituents other than —OH at C28 and/or substituents other than ═O at C30 may be used to obtain preferential binding to FRAP proteins bearing an amino acid substitution for Glu2032. Examplary mutations include E2032A and E2032S. Peptide sequence numbering and rapamycin numbering is with reference to WO 96/41865. Illustrative mutations in cyclophilin domains are disclosed in WO 94/18317 and may also be adapted for use in practicing the subject invention.

Design and Assembly of the DNA Constructs

Constructs may be designed in accordance with the principles, illustrative examples and materials and methods disclosed in the patent documents and scientific literature cited herein, each of which is incorporated herein by reference, with modifications and further exemplification as described herein. Components of the constructs can be prepared in conventional ways, where the coding sequences and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc. as appropriate. In the case of DNA constructs encoding chimeric proteins, DNA sequences encoding individual domains and sub-domains are joined such that they constitute a single open reading frame encoding a chimeric protein capable of being translated in cells or cell lysates into a single polypeptide harboring all component domains. The DNA construct encoding the chimeric protein may then be placed into a vector that directs the expression of the protein in the appropriate cell type(s). For biochemical analysis of the encoded chimera, it may be desirable to construct plasmids that direct the expression of the protein in bacteria or in reticulocyte-lysate systems. For use in the production of proteins in mammalian cells, the protein-encoding sequence is introduced into an expression vector that directs expression in these cells. Expression vectors suitable for such uses are well known in the art. Various sorts of such vectors are commercially available.

II. Delivery of DNA to the Mammal

Any means for the introduction of heterologous DNA into mammals, human or non-human, may be adapted to the practice of this invention for the delivery of the various DNA constructs into the intended recipient. For the purpose of this discussion, the various DNA constructs described herein (one or more DNA sequences encoding chimeric proteins under the control of a constitutive promoter such as a CMV promoter and one or more target gene constructs) may together be referred to as the transgene. Two general in vivo gene therapy approaches include (a) the delivery of “naked”, lipid-complexed or liposome-formulated or otherwise formulated DNA and (b) the delivery of the heterologous DNA via viral vectors. In the former approach, prior to formulation of DNA, e.g. with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See e.g. Canonico et al, Am J Respir Cell Mol Biol 10:24-29, 1994 (in vivo transfer of an aerosolized recombinant human alphal-antitrypsin gene complexed to cationic liposomes to the lungs of rabbits); Tsan et al, Am J Physiol 268 (Lung Cell Mol Physiol 12): L1052-L1056, 1995 (transfer of genes to rat lungs via tracheal insufflation of plasmid DNA alone or complexed with cationic liposomes); Alton et al., Nat Genet. 5:135-142, 1993 (gene transfer to mouse airways by nebulized delivery of cDNA-liposome complexes). Alternatively, the transgene may be incorporated into any of a variety of viral vectors useful in gene therapy. In either case, delivery of vectors or naked or formulated DNA can be carried out by instillation via bronchoscopy, after transfer of viral particles to Ringer's, phosphate buffered saline, or other similar vehicle, or by nebulization.

While various viral vectors may be used in the practice of this invention, AAV- and adenovirus-based approaches are of particular interest. The following additional guidance on the choice and use of viral vectors may be helpful to the practitioner.

### AAV Vectors

Adeno-associated virus (AAV)-based vectors are of general interest as a delivery vehicle to various tissues, including the lung. AAV vectors infect cells and stably integrate into the cellular genome with high frequency. AAV can infect and integrate into growth-arrested cells (such as the pulmonary epithelium), and is non-pathogenic.

The AAV-based expression vector to be used typically includes the 145 nucleotide AAV inverted terminal repeats (rrS) flanking a restriction site that can be used for subcloning of the transgene, either directly using the restriction site available, or by excision of the transgene with restriction enzymes followed by blunting of the ends, ligation of appropriate DNA linkers, restriction digestion, and ligation into the site between the ITRs. The capacity of AAV vectors is about 4.4 kb. The following proteins have been expressed using various AAV-based vectors, and a variety of promoter/enhancers: neomycin phosphotransferase, chloramphenicol acetyl transferase, Fanconi's anemia gene, cystic fibrosis transmembrane conductance regulator, and granulocyte macrophage colony-stimulating factor (Kotin, R. M., Human Gene Therapy 5:793-801, 1994, Table I). A transgene incorporating the various DNA constructs of this invention can similarly be included in an AAV-based vector. As an alternative to inclusion of a constitutive promoter such as CMV to drive expression of the recombinant DNA encoding the chimeric protein(s), e.g. chimeric proteins comprising an activation domain or DNA-binding domain, an AAV promoter can be used (ITR itself or AAV p5 (Flotte, et al. J. Biol. Chem. 268:3781-3790, 1993)).

Such a vector can be packaged into AAV virions by reported methods. For example, a human cell line such as 293 can be co-transfected with the AAV-based expression vector and another plasmid containing open reading frames encoding AAV rep and cap under the control of endogenous AAV promoters or a heterologous promoter. In the absence of helper virus, the rep proteins Rep68 and Rep78 prevent accumulation of the replicative form, but upon superinfection with adenovirus or herpes virus, these proteins permit replication from the ITRs (present only in the construct containing the transgene) and expression of the viral capsid proteins. This system results in packaging of the transgene DNA into AAV virions (Carter, B.J., Current Opinion in Biotechnology 3:533-539, 1992; Kotin, R. M, Human Gene Therapy 5:793-801, 1994)). Methods to improve the titer of AAV can also be used to express the transgene in an AAV virion. Such strategies include, but are not limited to: stable expression of the ITR-flanked transgene in a cell line followed by transfection with a second plasmid to direct viral packaging; use of a cell line that expresses AAV proteins inducibly, such as temperature-sensitive inducible expression or pharmacologically inducible expression. Additionally, one may increase the efficiency of AAV transduction by treating the cells with an agent that facilitates the conversion of the single stranded form to the double stranded form, as described in Wilson et al., WO96/39530.

Concentration and purification of the virus can be achieved by reported methods such as banding in cesium chloride gradients, as was used for the initial report of AAV vector expression in vivo (Flotte, et al. J. Biol. Chem. 268:3781-3790, 1993) or chromatographic purification, as described in O'Riordan et al., WO97/08298.

For additional detailed guidance on AAV technology which may be useful in the practice of the subject invention, including methods and materials for the incorporation of a transgene , the propagation and purification of the recombinant AAV vector containing the transgene, and its use in transfecting cells and mnammals, see e.g. Carter et al, U.S. Pat. No. 4,797,368 (Jan. 10, 1989); Muzyczka et al, U.S. Pat. No. 5,139,941 (Aug. 18, 1992); Lebkowski et al, U.S. Pat. No. 5,173,414 (Dec. 22, 1992); Srivastava, U.S. Pat. No. 5,252,479 (Oct. 12, 1993); Lebkowski et al, U.S. Pat. No. 5,354,678 (Oct. 11, 1994); Shenk et al, U.S. Pat. No. 5,436,146 (Jul. 25, 995); Chatterjee et al, U.S. Pat. No. 5,454,935 (Dec. 12, 1995), Carter et al WO 93/24641 (published Dec. 9, 1993), and Flotte et al., U.S. Pat. No. 5,658,776 (Aug. 19, 1997).

### Adenovirus Vectors

Various adenovirus vectors have been shown to be of use in the transfer of genes to mammals, including humans. Replication-deficient adenovirus vectors have been used to express marker proteins and CFIR in the pulmonary epithelium. Because of their ability to efficiently infect non-dividing cells, their tropism for the lung, and the relative ease of generation of high titer stocks, adenoviral vectors have been the subject of much research in the last few years, and various vectors have been used to deliver genes to the lungs of human subjects (Zabner et al., Cell 75:207-216, 1993; Crystal, et al., Nat Genet. 8:42-51, 1994; Boucher, et al., Hum Gene Ther 5:615-639, 1994). The first generation E1 deleted adenovirus vectors have been improved upon with a second generation that includes a temperature-sensitive E2a viral protein, designed to express less viral protein and thereby make the virally infected cell less of a target for the immune system (Goldman et al., Human Gene Therapy 6:839-851, 1995). More recently, a viral vector deleted of all viral open reading frames has been reported (Fisher et al., Virology 217:11-22, 1996). Moreover, it has been shown that expression of viral IL-10 inhibits the immune response to adenoviral antigen (Qin et al., Human Gene Therapy 8:1365-1374, 1997).

DNA sequences of a number of adenovirus types are available from Genbank. The adenovirus DNA sequences may be obtained from any of the 41 human adenovirus types currently identified. Various adenovirus strains are available from the American Type Culture Collection, Rockville, Md., or by request from a number of commercial and academic sources. A transgene as described herein may be incorporated into any adenoviral vector and delivery protocol, by the same methods (restriction digest, linker ligation or filling in of ends, and ligation) used to insert the CFrR or other genes into the vectors. Hybrid Adenovirus-AAV vectors represented by an adenovirus capsid containing selected portions of the adenovirus sequence, 5′ and 3′ AAV ITR sequences flanking the transgene and other conventional vector regulatory elements may also be used. See e.g. Wilson et al, International Patent Application Publication No. WO 96/13598. For additional detailed guidance on adenovirus and hybrid adenovirus-AAV technology which may be useful in the practice of the subject invention, including methods and materials for the incorporation of a transgene , the propagation and purification of recombinant virus containing the transgene, and its use in transfecting cells and mammals, see also Wilson et al, WO 94/28938, WO 96/13597 and WO 96/26285, and references cited therein.

Generally the DNA or viral particles are transferred to a biologically compatible solution or pharmaceutically acceptable delivery vehicle, such as sterile saline, or other aqueous or non-aqueous isotonic sterile injection solutions or suspensions, numerous examples of which are well known in the art, including Ringer's, phosphate buffered saline, or other similar vehicles. Delivery of the transgene as naked DNA; as lipid-, liposome-, or otherwise formulated DNA; or as a recombinant viral vector is then preferably carried out via in vivo, lung-directed, gene therapy. This can be accomplished by various means, induding nebulization/inhalation or by instillation via bronchoscopy. Recently, recombinant adenovirus encoding CFTR was administered via aerosol to human subjects in a phase I clinical trial. Vector DNA and CFTR expression were clearly detected in the nose and airway of these patients with no acute toxic effects (Bellon et al., Human Gene Therapy, 8(1):15-25, 1997).

Preferably, the DNA or recombinant virus is administered in sufficient amounts to transfect cells within the recipient's airways, including without limitation various airway epithelial cells, leukocytes residing within the airways and accessible airway smooth muscle cells, and provide sufficient levels of transgene expression to provide for observable ligand-responsive transcription of a target gene, preferably at a level providing therapeutic benefit without undue adverse effects. Optimal dosages of DNA or virus depends on a variety of factors, as discussed previously, and may thus vary somewhat from patient to patient. Again, therapeutically effective doses of viruses are considered to be in the range of about 20 to about 50 ml of saline solution containing concentrations of from about 1×10⁷ to about 1×10¹⁰ pfu of virus/ml, e.g. from 1×10⁸ to 1×10⁹ pfu of virus/ml.

By way of illustration, specific protocols for the administration of recombinant adenovirus via bronchoscope to baboons and to human patients are disclosed in Wilson et al WO 94/28938 which may be adapted to the general practice of the subject invention.

III. Pharmaceutical Compositions and Their Administration to Subjects Containing Engineered Cells

Administration of Ligands

A. In General

The ligand may be administered as desired using pharmaceutically acceptable materials and methods of administration. Depending upon factors such as the binding affinity of the ligand, the response desired, the manner/route of administration, the biological half-life and bioavailability of the ligand, the number of engineered cells present, etc. various protocols may be employed. The ligand may be administered parenterally, or more preferably orally. Dosage and frequency of administration will depend upon factors such as described above. The ligand may be taken orally as a pill, powder, or dispersion; bucally; sublingually; injected intravascularly, intraperitoneally, subcutaneously; or the like. The ligand (and antagonists, as discussed below) may be formulated using conventional methods and materials well known in the art for the various routes of administration. The precise dose and particular method of administration will depend upon the above factors and be determined by the attending physician or healthcare provider.

The particular dosage of the ligand for any application may be determined in accordance with conventional approaches and procedures for therapeutic dosage monitoring. A dose of the ligand within a predetermined range is given and the patient's response is monitored so that the level of therapeutic response and the relationship of target gene expression level over time may be determined. Depending on the expression levels observed during the time period and the therapeutic response, one may adjust the level of subsequent dosing to alter the resultant expression level over time or to otherwise improve the therapeutic response. This process may be iteratively repeated until the dosage is optimized for therapeutic response. Where the ligand is to be administered chronically, once a maintenance dosage of the ligand has been determined, one may conduct periodic follow-up monitoring to assure that the desired target gene expression level or overall therapeutic response continues to be achieved.

In the event that the activation by the ligand is to be reversed, administration of ligand may be suspended so that activation of transcription of the target gene(s) is allowed to return to its base line, which ideally represents little or no expression of the introduced target gene(s). To effect a more active reversal of therapy, an antagonist of the ligand may be administered. An antagonist is a compound which binds to the ligand or ligand-binding domain to inhibit interaction of the ligand with the chimeric protein(s) and thus inhibit the activation of target gene transcription. In embodiments which rely upon dimerization-based transcription activation, antagonists include ligand analogs, homologs or components which are monovalent with respect to the chimeric proteins. Such compounds bind to the chimeric proteins but do not support clustering of the chimeric proteins as is required for activation of transcription. Thus, in the case of an adverse reaction or the desire to terminate the therapeutic effect, an antagonist can be administered in any convenient way, particularly intravascularly or by inhalation/nebulization, if a rapid reversal is desired. Alternatively, using a different ligand, one may provide for the presence of a ligand-dependent transcription inhibition means analogous to the transcription activation means which is the focus of this document.

B. Therapeutic/Prophylactic Administration & Pharmaceutical Compositions

Ligands for use in this invention can exist in free form or, where appropriate, in salt form. The preparation of a wide variety of pharmaceutically acceptable salts is well-known to those of skill in the art. Pharmaceutically acceptable salts of various compounds include the conventional non-toxic salts or the quaternary ammonium salts of such compounds which are formed, for example, from inorganic or organic acids of bases.

The ligands may form hydrates or solvates. It is known to those of skill in the art that charged compounds form hydrated species when lyophilized with water, or form solvated species when concentrated in a solution with an appropriate organic solvent.

The ligands can also be administered as pharmaceutical compositions comprising a therapeutically (or prophylactically) effective amount of the ligand, and a pharmaceutically acceptable carrier or excipient. Carriers include e.g. saline, buffered saline, dextrose, water, glycerol, ethanol, and combinations thereof, and are discussed in greater detail below. The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Formulation may involve mixing, granulating and compressing or dissolving the ingredients as appropriate to the desired preparation.

The pharmaceutical carrier employed may be, for example, either a solid or liquid.

Illustrative solid carriers include lactose, terra alba, sucrose, talc, gelatin, agar, pectin, acacia, magnesium stearate, stearic acid and the like. A solid carrier can include one or more substances which may also act as flavoring agents, lubricants, solubilizers, suspending agents, fillers, glidants, compression aids, binders or tablet-disintegrating agents; it can also be an encapsulating material. In powders, the carrier is a finely divided solid which is in admixture with the finely divided active ingredient. In tablets, the active ingredient is mixed with a carrier having the necessary compression properties in suitable proportions, and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyinylpyrrolidine, low melting waxes and ion exchange resins.

Illustrative liquid carriers include syrup, peanut oil, olive oil, water, etc. Liquid carriers are used in preparing solutions, suspensions, emulsions, syrups, elixirs and pressurized compositions. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate and isopropyl myristate. Sterile liquid carders are useful in sterile liquid form compositions for parenteral administration. The liquid carrier for pressurized compositions can be halogenated hydrocarbon or other pharmaceutically acceptable propellant. Liquid pharmaceutical compositions which are sterile solutions or suspensions can be utilized by, for example, intramuscular, intraperitoneal or subcutaneous injection. Sterile solutions can also be administered intravenously. The ligands can also be administered orally either in liquid or solid composition form.

The carrier or excipient may include time delay material well known to the art, such as glyceryl monostearate or glyceryl distearate along or with a wax, ethylcellulose, hydroxypropylmethylcellulose, methylmethacrylate and the like. When formulated for oral administration, 0.01% Tween 80 in PHOSAL PG-50 (phospholipid concentrate with 1,2-propylene glycol, A. Nattermann & Cie. GmbH) may be used as an oral formulation for a variety of ligands for use in the practice of this invention.

A wide variety of pharmaceutical forms can be employed. If a solid carrier is used, the preparation can be tableted, placed in a hard gelatin capsule in powder or pellet form or in the form of a troche or lozenge. The amount of solid carrier will vary widely but preferably will be from about 25 mg to about 1 g. If a liquid carrier is used, the preparation will be in the form of a syrup, emulsion, soft gelatin capsule, sterile injectable solution or suspension in an ampule or vial or nonaqueous liquid suspension.

To obtain a stable water soluble dosage form, a pharmaceutically acceptable salt of the ligand may be dissolved in an aqueous solution of an organic or inorganic acid, such as a 0.3M solution of succinic acid or citric acid. Alternatively, acidic derivatives can be dissolved in suitable basic solutions. If a soluble salt form is not available, the compound is dissolved in a suitable cosolvent or combinations thereof. Examples of such suitable cosolvents include, but are not limited to, alcohol, propylene glycol, polyethylene glycol 300, polysorbate 80, glycerin, polyoxyethylated fatty acids, fatty alcohols or glycerin hydroxy fatty acids esters and the like in concentrations ranging from 0-60% of the total volume.

Various delivery systems are known and can be used to administer the ligands, or the various formulations thereof, including tablets, capsules, injectable solutions, encapsulation in liposomes, microparticles, microcapsules, etc. Preferred routes of administration are oral, sublingual and bucal. Methods of introduction also could include but are not limited to dermal, intradermal, intramuscular, intraperitoneal, intravenous, subcutaneous, intranasal, pulmonary, epidural, ocular and (as is usually preferred) oral routes. The ligand may be administered by any convenient or otherwise appropriate route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic to ease pain at the side of the injection. Generally, the ingredients are supplied either separately or mixed together in unit dosage form, for example, as a lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In addition, in certain instances, it is expected that the compound may be disposed within devices placed upon, in, or under the skin. Such devices include patches, implants, and injections which release the compound into the skin, by either passive or active release mechanisms.

Materials and methods for producing the various formulations are-well known in the art and may be adapted for practicing the subject invention. See e.g. U.S. Pat. Nos. 5,182,293 and 4,837,311 (tablets, capsules and other oral formulations as well as intravenous formulations) and European Patent Application Publication Nos. 0 649 659 (published Apr. 26, 1995; rapamycin formulation for IV administration) and 0 648 494 (published Apr. 19, 1995; rapamycin formulation for oral administration).

The effective dose of the ligand will typically be in the range of about 0.01 to about 50 mg/kgs, preferably about 0.1 to about 10 mg/kg of mammalian body weight, administered in single or multiple doses. Generally, the compound may be administered to patients in need of such treatment in a daily dose range of about 1 to about 2000 mg per patient. In embodiments in which the compound is rapamycin or an analog thereof with some residual immunosuppressive effects, it is preferred that the dose administered be below that associated with undue immunosuppressive effects.

The amount of a given ligand which will be effective in the treatment or prevention of a particular disorder or condition will depend in part on the severity of the disorder or condition, and can be determined by standard clinical techniques. In addition, in vitro or in vivo assays may optionally be employed to help identify optimal dosage ranges. Effective doses may be extrapolated from dose-response curves derived from in vitro or animal model test systems. The precise dosage level should be determined by the attending physician or other health care provider and will depend upon well known factors, including route of administration, and the age, body weight, sex and general health of the individual; the nature, severity and clinical stage of the disease; the use (or not) of concomitant therapies; and the nature and extent of genetic engineering of cells in the patient.

The ligands can also be provided in a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceutical or biological products, which notice reflects approval by the agency of manufacture, use or sale for human administration.

IV. In vivo Studies in Animals

Animal models of atopic asthma have been established as reproducible and useful. In such models, animals can be sensitized to a defined antigen, leading to increases in IgE and IgG titers in the animals. Subsequent exposure to aerosolized antigen yields the three conditions apparent in human asthma 1) acute bronchoconstriction, 2) pulmonary inflammation, and 3) airway hyperreactivity.

Mice provide a good model because different strains exhibit variations of the pathophysiologic sequelae of this pulmonary atopic response. Immunologic reagents and tools are available to evaluate these sequelae in nice.

Guinea pigs may also be used due to their seroconversion from IgG to IgE, along with the high titers, much as is seen in humans. Rats may also be used. Rabbits, dogs, sheep, and non-human primates may be used as models for naturally occurring “asthma” and airway hyperreactivity. These models are most like human asthma. The animals can be naturally sensitized to some defined antigen that must be discovered (it is often Ascaris suum). This sensitivity should then cause underlying airway hyperreactivity, pulmonary inflammation, and acute bronchoconstriction.

Such sensitization procedures and pulmonary function tests are well described in the literature and are commonly used in drug development.

V. Illustrative Embodiments

The examples which follow illustrate the design, production and use of target gene constructs. Also illustrated are DNA constructs encoding various chimeric proteins useful for the direct and indirect activation of transcription of a target gene in a divalent ligand-dependant manner. Protocols are also set forth for determining the efficacy of transfection in mammals using bronchoalveolar lavage and for determining the efficacy of the overall therapy by monitoring various pulmonary functions.

### EXAMPLES

### Cellular Transformations and Evaluation

### Example 1

### Induction of Transcription by Cross-Linking the CD3 Chain of the T-Cell Receptor

The plasmid pSXNeo/IL2 (IL2-SX) (FIG. 1 of PCT/US94/01617), which contains the placental secreted alkaline phosphatase gene under the control of human IL-2 promoter (−325 to +47; MCB(86) 6, 3042), and related plasmid variants (i.e. NFAT-SX, NFB-SX, OAP/Octl-SX, and AP-1-SX) in which the reporter gene is under the transcriptional control of the minimal IL-2 promoter (−325 to −294 and −72 to +47) combined with synthetic oligomers containing various promoter elements (i.e. NFAT, NKB, OAP/Oct-1, and AP1, respectively), were made by three piece ligations of 1) pPL/SEAP (Berger, et al., Gene (1988) 66,1) cut with Sspl and HindIII; 2) pSV2/Neo (Southern and Berg, J. Mol. Appl. Genet. (1982) 1, 332) cut with NdeI, blunted with Kilenow, then cut with PvuI; and 3) various promoter-containing plasmids (i.e. NFAT-CD8, B-CD8, cxl2lacZ-Oct-1, AP1-LUCIF3H, or cx151L2) (described below) cut with PvuI and HindIII. NFAT-CD8 contains 3 copies of the NFAT-binding site (−286 to −257; Genes and Dev. (1990) 4, 1823) and cx121acZ-Oct contains 4 copies of the OAP/Oct-1/(ARRE-1) binding site (MCB, (1988) 8, 1715) from the human IL-2 enhancer; B-CD8 contains 3 copies of the NFB binding site from the murine light chain (EMBO (1990) 9,4425) and AP1-LUCIF3H contains 5 copies of the AP-1 site (5′-TGACTCAGCGC-3′) from the metallothionen promoter.

In each transfection, 5 μg of expression vector, pCDL-SR (MCB 8, 466-72) (Tac-IL2 receptor-chain), encoding the chimeric receptor TAC/TAC/Z (TTZ) (PNAS 88, 8905-8909), was co-transfected along with various secreted alkaline phosphatase-based reporter plasmids (see map of pSXNeo/IL2 in FIG. 1 of PCT/US94/01617) in TAg Jurkat cells (a derivative of the human T-cell leukemia line Jurkat stably transfected with the SV40 large T antigen (Northrup, et al., J. Biol. Chem. [1993]). Each reporter plasmid contains a multimerized oligonucleotide of the binding site for a distinct IL-2 enhancer-binding transcription factor within the context of the minimal IL-2 promoter or, alternatively, the intact IL-2 enhancer/promoter upstream of the reporter gene. After 24 hours, aliquots of cells (approximately 10⁵) were placed in microtiter wells containing log dilutions of bound anti-TAC (CD25) mAb (33B3.1; AMAC, Westbrook, Me.). As a positive control and to control for transfection efficiency, ionomycin (1 m) and PMA (25 ng/ml) were added to aliquots from each transfection. After an additional 14 hour incubation, the supernatants were assayed for the alkaline phosphatase activity and these activities were expressed relative to that of the positive control samples. The addition of 1 ng/ml FK506 dropped all activity due to NFAT to background levels, demonstrating that deactivations are in the same pathway as that blocked by FK506. Each data point obtained was the average of two samples and the experiment was performed several times with similar results. See FIG. 5 of PCT/US94/01617. The data show that with a known extracellular receptor, one obtains an appropriate response with a reporter gene and different enhancers. Similar results were obtained when a MAb against the TcR complex (i.e. OKT3) was employed.

### Example 2

### Activity of the Dimeric FK506 Derivative, FK1012A, on the Chimeric FKBP12/CD3 (1FK3) Receptor

5 μg of the eukaryotic expression vector, pBJ5, (based on pCDL-SR with a polylinker inserted between the 16S splice site and the poly A site), containing the chimeric receptor (1FK3), was co-transfected with 4 μg of the NFAT-inducible secreted alkaline phosphatase reporter plasmid, NFAT-SX. As a control, 5 μg of pBJ5 was used, instead of 1FK3/pBJ5, in a parallel transfection. After 24 hours, aliquots of each transfection containing approximately 10⁵ cells were incubated with log dilutions of the drug FK1012A, as indicated. As a positive control and to control for transfection efficiency, ionomycin (1 μM) and PMA (25 ng/ml) were added to aliquots from each transfection. After an additional 14 hour incubation, the supernatants were assayed for alkaline phosphatase activity and these activities were expressed relative to that of the positive control samples. The addition of 2 ng/ml FK506 dropped all stimulations to background levels, demonstrating that the activations are in the same pathway as that blocked by FK506. Hence, FK506 or cyclosporin will serve as effective antidotes to the use of these compounds. Each data point obtained was the average of two samples and the experiment was performed several times with similar results. See FIG. 7 of PCT/US94/01617.

### Example 3

### Activity of the Dimeric FK506 Derivative, FK1012B, on the Myristoylated Chimeric CD3/FKBP12 (MZF3E) Receptor

A number of approaches to ligand design and synthesis have been successfully demonstrated, e.g., in WO 94/18317, including positive results with FK506-based homodimeric reagents named “FK1012”s. FK1012s were found to achieve high affinity, 2:1 binding stoichiometry (K_{d}(1)=0.1 nM; K_{d}(2)=0.8 nM) and were found to not inhibit calcineurin-mediated TCR signaling. The ligands are neither “immunosuppressive” nor toxic (up to 0.1 mM in cell culture). Similarly, a cyclosporin A-based homodimerizing agent, “(CsA)2” was prepared which binds to the CsA receptor, cyclophylin, with 1:2 stoichiometry, but which does not bind to calcineurin. Thus, like FK1012s, (CsA)₂ does not inhibit signalling pathways and is thus neither inununosuppressive nor toxic.

These and other examples of ligand-mediated protein association resulted in the control of a signal transduction pathway. In an illustrative case, this was accomplished by creating an intracellular receptor comprised of a small fragment of Src sufficient for posttranslational myristoylation (M), the cytoplasmic tail of zeta (Z; a component of the B cell receptor was also used), three consecutive FKBP12s (F3) and a flu epitope tag (E). Expression of the construct MZF3E (FIG. 18 of PCT/US94/01617) in human (Jurkat) T cells confirmed that the encoded chimeric protein underwent FK1012-mediated oligomerization. The attendant aggregation of the zeta chains led to signaling via the endogenous TCR-signaling pathway (FIG. 15 of PCT/US94/01617), as evidenced by secretion of alkaline phosphatase (SEAP) in response to an FK1012 (EC₅₀=50 nM). The promoter of the SEAP reporter gene was constructed to be transcriptionally activated by nuclear factor of activated T cells (NFAT), which is assembled in the nucleus following TCR-signaling. FK1012-induced signaling can be terminated by a deaggregation process induced by a nontoxic, monomeric version of the ligand called FK506-M.

Specifically, 5 μg of the eukaryotic expression vector, pBJ5, containing a myristoylated chimeric receptor was co-transfected with 4 μg NFAT-SX. MZE, MZF1E, MZF2E and MZF3E contain 0, 1, 2, or 3 copies of FKBP12, respectively, downstream of a myristoylated CD3 cytoplasmic domain (see FIG. 2 of PCT/US94/01617). As a control, 5 μg of pBJ5 was used in a parallel transfection. After 24 hours, aliquots of each transfection containing approximately 10⁵ cells were incubated with log dilutions of the drug, FK1012B, as indicated. As a positive control and to control for transfection efficiency, ionomycin (1 μm) and PMA (25 ng/ml) were added to aliquots from each transfection. After an additional 12 hour incubation, the supernatants were assayed for alkaline phosphatase activity and these activities were expressed relative to that of the positive control samples. The addition of 1 ng/ml FK506 dropped all stimulations to near background levels, demonstrating that the activations are in the same pathway as that blocked by FK506. This result is further evidence of the reversibility of the subject cell activation. Each data point obtained was the average of two samples and the experiment was performed several times with similar results. See FIG. 8 of PCT/US94/01617. The myristoylated derivatives respond to lower concentrations of the ligand by about an order of magnitude and activate NF-AT dependent transcription to comparable levels, but it should be noted that the ligands are different. Compare FIGS. 7 and 8 of PCT/US94/01617.

In vivo FK1012-Induced Protein Dimerization

The following experiments confirmed that intracellular aggregation of the MZF3E receptor is indeed induced by the FK1012. The influenza haemagglutinin epitope-tag (flu) of the MZF3E-construct was exchanged with a different epitope-tag (flag-M2). The closely related chimeras, MZF3E_{flu} and MZF3E_{flag}, were coexpressed in Jurkat T cells. Immunoprecipitation experiments using anti-Flag-antibodies coupled to agarose beads were performed after the cells were treated with FK1012A. In the presence of FK1012A (1 μM) the protein chimera MZF³E_{flag} interacts with MZF3E_{flu} and is coimmunoprecipitated with MZF³E_{flag}. In absence of FK1012A, no coimmunoprecipitation of MZF3E_{flu} is observed. Related experiments with FKBP monomer constructs MZF1E_{flu} and MZF1E_{flag}, which do not signal, revealed that they are also dimerized by FK1012A (FIG. 19A of PCT/US94/01617). This reflects the requirement for aggregation observed with both the endogenous T cell receptor and our artificial receptor MZF3E.

FK1012-Induced Protein-Tyrosine Phosphorylation

The intracellular domains of the TCR, CD3 and zeta-chains interact with cytoplasmic protein tyrosine kinases following antigen stimulation. Specific members of the Src family (lck and/or fyn) phosphorylate one or more tyrosine residues of activation motifs within these intracellular domains (tyrosine activation motif, TAM). The tyrosine kinase ZAP-70 is recruited (via its two SH2 domains) to the tyrosine phosphorylated T-cell-receptor, activated, and is likely to be involved in the further downstream activation of phospholipase C. Addition of either anti-CD3 MAb or FK1012A to Jurkat cells stably transfected with MZF3E resulted in the recruitment of kinase activity to the zeta-chain as measured by an in vitro kinase assay following immunoprecipitation of the endogenous T cell receptor zeta chain and the MZF3E-construct, respectively. Tyrosine phosphorylation after treatment of cells with either anti-CD3 MAb or FK1012 was detected using monoclonal alpha-phosphotyrosine antibodies. Whole cell lysates were analysed at varying times after stimulation. A similar pattern of tyrosine-phosphorylated proteins was observed after stimulation with either anti-CD3 MAb or FK1012. The pattern consisted of a major band of 70 kDa, probably ZAP-70, and minor bands of 120 kDa, 62 kDa, 55 kDa and 42 kDa.

### Example 4

### Construction of Murine Signalling Chimeric Protein

The various fragments were obtained by using primers described in FIG. 4 of PCT/US94/01617. In referring to primer numbers, reference should be made to FIG. 4 of PCT/US94/01617.

An approximately 1.2 kb cDNA fragment comprising the I-E chain of the murine class II MHC receptor (Cell, 32, 745) was used as a source of the signal peptide, employing P#6048 and P#6049 to give a 70 bp SacII-XhoI fragment using PCR as described by the supplier (Promega). A second fragment was obtained using a plasmid comprising Tac (IL2 receptor chain) joined to the transmembrane and cytoplasmic domains of CD3 (PNAS, 88, 8905). Using P#6050 and P#6051, a 320 bp XhoI-EcoRI fragment was obtained by PCR comprising the transmembrane and cytoplasmic domains of CD3. These two fragments were ligated and inserted into a SacII-EcoRI digested pBluescript (Stratagene) to provide plasmid, SPZ/KS.

To obtain the binding domain for FK506, plasmid rhFKBP (provided by S. Schreiber, Nature (1990) 346, 674) was used with P#6052 and P#6053 to obtain a 340 bp XhoI-SalI fragment containing human FKBP12. This fragment was inserted into pBluescript digested with XhoI and SalI to provide plasmid FK12/KS, which was the source for the FKBP12 binding domain. SPZ/KS was digested with XhoI, phosphatased (cell intestinal alkaline phosphatase; CIP) to prevent self-annealing, and combined with a 10-fold molar excess of the XhoI-SalI FKBP12-containing fragment from FK12/KS. Clones were isolated that contained monomers, dimers, and trimers of FKBP12 in the correct orientation. The clones 1FK1/KS, 1FK2/KS, and 1FK3/KS are comprised of in the direction of transcription; the signal peptide from the murine MHC class II gene I-E, a monomer, dimer or trimer, respectively, of human FKBP12, and the transmembrane and cytoplasmic portions of CD3. Lastly, the SacII-EcoRI fragments were excised from pBluescript using restriction enzymes and ligated into the polylinker of pBJ5 digested with SacII and EcoRI to create plasmids 1FK1/pBJ5, 1FK2/pBJ5, and 1FK3/pBJ5, respectively. See FIGS. 3 and 4 of PCT/US94/01617.

### Example 5

A. Construction of Intracellular Signaling Chimera.

A myristoylation sequence from c-src was obtained from Pellman, et al., Nature 314, 374, and joined to a complementary sequence of CD3 to provide a primer which was complementary to a sequence 3′ of the transmembrane domain, namely P#8908. This primer has a SacII site adjacent to the 5′ terminus and a XhoI sequence adjacent to the 3′ terminus of the myristoylation sequence. The other primer P#8462 has a SalI recognition site 3′ of the sequence complementary to the 3′ terminus of CD3, a stop codon and an EcoRI recognition site. Using PCR, a 450 bp SacII-EcoRI fragment was obtained, which was comprised of the myristoylation sequence and the CD3 sequence fused in the 5′ to 3′ direction. This fragment was ligated into SacII/EcoRI-digested pBJ5(XhoI)(SalI) and cloned, resulting in plasmid MZ/pBJ5. Lastly, MZ/pBJ5 was digested with SalI, phosphatased, and combined with a 10-fold molar excess of the XhoI-SalI FKBP12-containing fragment from FK12/KS and ligated. After cloning, the plasmids comprising the desired constructs having the myristoylation sequence, CD3 and FKBP12 multimers in the 5′-3′ direction were isolated and verified as having the correct structure. See FIGS. 2 and 4 of PCT/US94/01617.

B. Construction of Expression Cassettes for Intracellular Signaling Chimeras

The construct MZ/pBJ5 (MZE/pBJ5) is digested with restriction enzymes XhoI and SalI, the TCR fragment is removed and the resulting vector is ligated with a 10 fold excess of a monomer, dimer, trimer or higher order multimer of FKBP12 to make MF1E, MF2E, MF3E or MFₙE/pBJ5. Active domains designed to contain compatible flanking restriction sites (i.e. XhoI and SalI) can then be cloned into the unique XhoI or SalI restriction sites of MFₙE/pBJ5.

### Example 6

### Construction of Nuclear Chimera

A. GAL4 DNA Binding Domain—FKBP Domain(s)—Epitope Tag.

The GAL4 DNA binding domain (amino adds 1-147) was amplified by PCR using a 5′ primer (#37) that contains a SacI site upstream of a Kozak sequence and a translational start site, and a 3′ primer (#38) that contains a SalI site. The PCR product was isolated, digested with SacII and SalI, and ligated into pBluescript II KS (+) at the SacII and SalI Sites, generating the construct pBS-GAL4. The construct was verified by sequencing. The SacH/SalI fragment from pBS-GAL4 was isolated and ligated into the IFK1/pBJ5 and IFK3/pBJ5 constructs (containing the myristoylation sequence, see Example 5) at the SacII and XhoI sites, generating constructs GF1E, GF2E and GF3E.

5′ end of PCR Amplified Product:

```
5′ end of PCR amplified product:
        SacII |----Gal4(1-147)--->>
                                        M K L L S S I
5′ CGA\overline{CACCGC}GGCCACCATGAAGCTACTGTCTTCTATCG
                          \overline{ } Kozak
3′ end of PCR amplified product:
      <<----Gal4 (1-147----) |
      R Q L T V S
5′ GACAGTTGACTGTATCGGTCGACTGTCG
3′ CTGTCAACTGACATAGCCAGCTGACAGC
                            \overline{ SalI }
```

B. HNF1 Dimerization/DNA Binding Domain—FKBP Domain(s)—Tag.

The HNF1a dimerization/DNA binding domain (amino acids 1-282) was amplified by PCR using a 5′ primer (#39) that contains a SacII site upstream of a Kozak sequence and a translational start site, and a 3′ primer (#40) that contains a SalI site. The PCR product was isolated, digested with SacII and SalI, and ligated into pBluescript II KS (+) at the SacII and SalI sites, generating the construct pBS-HNF. The construct was verified by sequencing. The SacII/SalI fragment from pBS-HNF was isolated and ligated into the IFK1/pBJ5 and IFK3/pBJ5 constructs at the SacII and XhoI sites, generating constructs HF1E, HF2E and HF3E.

5′ end of PCR Amplified Product:

```
5′ end of PCR amplified product:
SacII |--HNF1 (1-281)-->>
                                    M V S K L S
5′ CG\overline{ACACCG}CGGCCACCATGGTTTCTAAGCTGAGC
                  \overline{Koza}k
3′ end of PCR amplified product:
      <<-------HNF1 (1-282) ------|
      A F R H K L
5′ CCTTCCGGCACAAGTTGGTCGACTGTCG
3′ GGAAGGCCGTGTTCAACCAGCTGACAGC
                          \overline{ SalI}
```

C. FKBP Domain(s)-VP16 Transcrip. Activation Domain(s)-Epitope Tag.

These constructs were made in three steps: (i) a construct was created from IFK3/pBJ5 in which the myristoylation sequence was replaced by a start site immediately upstream of an XhoI site, generating construct SF3E; (ii) a nuclear localization sequence was inserted into the XhoI site, generating construct NF3E; (iii) the VP16 activation domain was cloned into the SalI site of NF3E, generating construct NF3V1E.

(i). Complementary oligonucleotides (#45 and #46) encoding a Kozak sequence and start site flanked by SacH and XhoI sites were annealed, phosphorylated and ligated into the SacII and XhoI site of MF3E, generating construct SF3E.

Insertion of Generic Start Site

```
                Kozak
                        M L E
5′ GG\overline{CCACC}ATGC
3′ CGCCGGTGGTACGAGCT
      \overline{Sa}cII \overline{XhoI}
      overhang overhang
```

(ii). Complementary oligonucleotides (#47 and #48) encoding the SV40 T antigen nuclear localization sequence flanked by a 5′ SalI site and a 3′ XhoI site were annealed, phosphorylated and ligated into the XhoI site of SF1E, generating the construct NF1E. The construct was verified by DNA sequencing. A construct containing the mutant or defective form of the nuclear localization sequence, in which a threonine is substituted for the lysine at position 128, was also isolated. This is designated NFLE-M. Multimers of the FKBP12 domain were obtained by isolating the FKBP12 sequence as an XhoI/SalI fragment from pBS-FKBP12 and ligating this fragment into NF1E linearized with XkoI. This resulted in the generation of the constructs NF2E and NF3B.

Insertion of NLS into Generic Start Site

```
                          T (ACN)
                  126 132
      L D P K K K R K V L E
5′ TCGACCCTAAGAAGAAGAGAAAGGTAC
3′ GGGATTCTTCTTCTCTTTCCATGAGCT
      \overline{SalI } \overline{XhoI }
```

Threonine at position 128 results in a defective NLS.

(iii). The VP16 transcription activation domain (amino acids 413-490) was amplified by PCR using a 5′ primer (#43) that contains SalI site and a 3′ primer (#44) that contains an XhoI site. The PCR product was isolated, digested with SalI and XhoI, and ligated into MF3E at the XhoI and SalI sites, generating the construct MV1E. The construct was verified by sequencing. Multimerized VP16 domains were created by isolating the single VP16 sequence as a XhoI/Sail fragment from MV1E and ligating this fragment into MV1E linearized with XhoI. Constructs MV2E, MV3E and MV4E were generated in this manner. DNA fragments encoding one or more multiple VP16 domains were isolated as XhoI/SalI fragments from MV1E or MV2E and ligated into NF1E linearized with SalI, generating the constructs NFlVLE and NF1V3E. Multimers of the FKBP12 domain were obtained by isolating the FKBP12 sequence as an XhoI/SalI fragment from pBS-FKBP12 and ligating this fragment into NF1V1E linearized with XhoI. This resulted in the generation of the constructs NF2V1E and NF3V1E.

5′ end of PCR Amplified Product:

```
5′ end of PCR amplified product:
        SalI |--VP16(413-490)--->>
                      A P P T D V
5′ CGAC\overline{AGTC}GACGCCCCCCCGACCGATGTC
3′ end of PCR amplified product:
  <<--VP16(413-490)----|
            D E Y G G
5′ GACGAGTACGGTGGGCTCGAGTGTCG
3′ CTGCTCATGCCACCCGAGCTCACAGC
                                  \overline{Xho1 }
```

Oligonucleotides:

| | |
|---|---|
| #37 | 38 mer/0.2 um/OFF |

5′CGACACCGCGGCCACCATGAAGCTACTGTCTTCTA TCG

| | |
|---|---|
| #38 | 28 mer/0.2 um/OFF |

5′CGACAGTCGACCGATACAGTCAACTGTC

| | |
|---|---|
| #39 | 34 mer/0.2 um/OFF |

5′CGACACCGCGGCCACCATGGTTTCTAAGCTGAGC

| | |
|---|---|
| #40 | 28 mer/0.2 um/OFF |

5′CGACAGTCGACCAACTTGTGCCGGAAGG

| | |
|---|---|
| #43 | 29 mer/0.2 um/OFF |

5′CGACAGTCGACGCCCCCCCGACCGATGTC

| | |
|---|---|
| #44 | 26 mer/0.2 um/OFF |

5′CGACACTCGAGCCCACCGTACTCGTC

| | |
|---|---|
| #45 | 26 mer/0.2 um/OFF |

5′GGCCACCATGC

| | |
|---|---|
| #46 | 18 mer/0.2 um/OFF |

5′TCGAGCATGGTGGCCGC

| | |
|---|---|
| #47 | 27 mer/0.2 um/OFF |

5′TCGACCCTAAGA-(C/A)-GAAGAGAAAGGTAC

| | |
|---|---|
| #48 | 27 mer/0.2 um/OFF |

5′TCGAGTACCTTTCTCTTC-(G/T)-TCTTAGGG

### Example 7

The following additional examples illustrate chimeric proteins containing the composite DNA-binding domain ZFHD1 (See Pomerantz et al., 1995, Science 267:93-96) together with various other domains, and the use of these chimeras in constitutive and ligand-dependent transcriptional activation.

A. Plasmids

pCGNNZFHD1

An expression vector for directing the expression of ZFHD1 coding sequence in mammalian cells was prepared as follows. Zif268 sequences were amplified from a cDNA done by PCR using primers 5′Xba/Zif and 3′Zif+G. Oct1 homeodomain sequences were amplified from a cDNA clone by PCR using primers 5′Not Oct HD and Spe/Bam 3′Oct. The Zif268 PCR fragment was cut with XbaI and NotI. The OctI PCR fragment was cut with NotI and BamHI. Both fragments were ligated in a 3-way ligation between the XbaI and BamHI sites of pCGNN (Attar and Gilman, 1992) to make pCGNNZFHD1 in which the cDNA insert is under the transcriptional control of human CMV promoter and enhancer sequences and is linked to the nuclear localization sequence from SV40 T antigen. The plasmid pCGNN also contains a gene for ampicillin resistance which can serve as a selectable marker.

pCGNNZFHD1-p65

An expression vector for directing the expression in mammalian cells of a chimeric transcription factor containing the composite DNA-binding domain, ZFHD1, and a transcription activation domain from p65 (human) was prepared as follows. The sequence encoding the C-terminal region of p65 containing the activation domain (amino acid residues 450-550) was amplified from pCGN-p65 using primers p65 5′ Xba and p65 3′ Spe/Bam. The PCR fragment was digested with XbaI and BamH1 and ligated between the the Spe1 and BamH1 sites of pCGNN ZFHD1 to form pCGNN ZFHD-p65AD.

The P65 transcription activation sequence contains the following linear sequence:

CTGGGGGCCTTGCTTGGCAACAGCACAGACCCAGCTGTGTTCACAGACCT
GGCATCCGTCGACAACTCCGAGTTTCAGCAGCTGCTGAACCAGGGCATAC
CTGTGGCCCCCCACACAACTGAGCCCATGCTGATGGAGTACCCTGAGGCT
ATAACTCGCCTAGTGACAGGGGCCCAGAGGCCCCCCGACCCAGCTCCTGC
TCCACTGGGGGCCCCGGGGCTCCCCAATGGCCTCCTTTCAGGAGATGAAG
ACTTCTCCTCCATTGCGGACATGGACTTCTCAGCCCTGCTGAGTCAGATC
AGCTCC

pCGNNZFHDI-FKBPx3

An expression vector for directing the expression of ZFHD1 linked to three tandem repeats of human FKBP was prepared as follows. Three tandem repeats of human FKBP were isolated as an XbaI-BamHI fragment from pCGNNF3 and ligated between the Spe1 and BamHI sites of pCGNNZFHD1 to make pCGNNZFHD1-FKBPx3 (ATCC Accession No. 97399).

pZHWTx8SVSEAP

A reporter gene construct containing eight tandem copies of a ZFHD1 binding site (Pomerantz et al., 1995) and a gene encoding secreted alkaline phosphatase (SEAP) was prepared by ligating the tandem ZFHD1 binding sites between the Nhe1 and BglII sites of pSEAP-Promoter Vector (Clontech) to form pZHWTx8SVSEAP. The ZHWTx8SEAP reporter contains two copies of the following sequence in tandem:

CTAGCTAATGATGGGCGCTCGAGTAATGATGGGCGGTCGACTAATGATGG
GCGCTCGAGTAATGA TGGGCGT

The ZFHD1 binding sites are underlined.

pCGNN F1 and F2

One or two copies of FKBP12 were amplified from pNF3VE using primers FKBP 5′ Xba and FKBP 3′ Spe/Bam. The PCR fragments were digested with XbaI and BamHI and ligated between the XbaI and BamH1 sites of pCGNN vector to make pCGNN F1 or pPCGNN F2. pCGNNZFHD1-FKBPx3 can serve as an alternate source of the FKBP cDNA.

pCGNN F3

A fragment containing two tandem copies of FKBP was excised from pCGNN F2 by digesting with Xba1 and BamH1. This fragment was ligated between the Spe1 and BamH1 sites of pCGNN F1.

pCGNN F3VP16

The C-terminal region of the Herpes Simplex Virus protein, VP16 (AA 418-490) containing the activation domain was amplified from pCG-a14-VP16 using primers VP16 5′ Xba and VP16 3′ Spe/Bam. The PCR fragment was digested with XbaI and BamHI and ligated between the Spe1 and BamH1 sites of pCGNN F3 plasmid.

pCGNN F3p65

The Xba1 and BamH1 fragment of p65 containing the activation domain was prepared as described above. This fragment was ligated between the Spe1 and BamH1 sites of pCGNN F3.

| B. Primers | |
|---|---|
| 5′Xba/Zif | 5′ATGCTCTAGAGAACGCCCATATGCTTGCCCT |
| 3′Zif + G | 5′ATGCGCGGCCGCCGCCTGTGTGGGTGCGGATGTG |
| 5′Not OctHD | 5′ATGCGCGGCCGCAGGAGGAAGAAACGCACCAGC |
| Spe/Bam 3′Oct | 5′GCATGGATCCGATTCAACTAGTGTTGATTCTTTTTTCTTTCTGGCGGCG |
| FKBP 5′Xba | 5′TCAGTCTAGAGGAGTGCAGGTGGAAACCAT |
| FKBP 3′Spe/Bam | 5′TCAGGGATCCTCAATAACTAGTTTCCAGTTTTAGAAGCTC |
| VP16 5′Xba | 5′ACTGTCTAGAGTCAGCCTGGGGGACGAG |
| VP16 3′Spe/Bam | 5′GCATGGATCCGATTCAACTAGTCCCACCGTACTCGTCAATTCC |
| P65 5′Xba | 5′ATGCTCTAGACTGGGGGCCTTGCTTGGCAAC |
| p65 3′Spe/Bam | 5′GCATGGATCCGCTCAACTAGTGGAGCTGATCTGACTCAG |

C. Dimerizing Agent

FK1012 consists of two molecules of the natural product FK506covalently joined to one another by a synthetic linker and can be prepared from FK506 using published procedures. See e.g. PCT/US94/01617 and Spencer et al, 1993. FK1012 is capable of binding to two FKBP domains and functioning as a dimerizing agent for FKBP-containing chimeric proteins. “FK1012” without further qualification generally refers to FK1012A, although various FK1012 species may be used (see e.g. structural representation below and PCT/US94/01617). PK506-M is included here only as an illustrative example of an antagonist of the divalent FK1012 ligands:

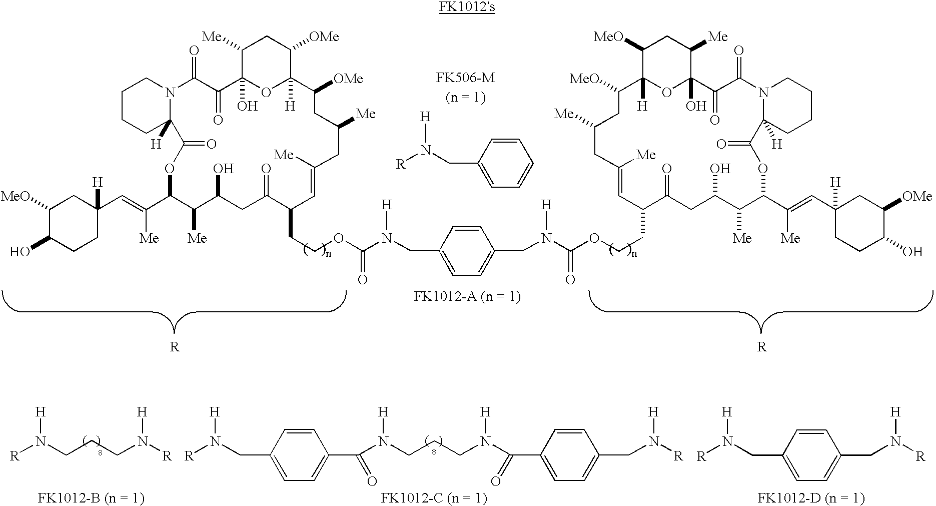

(i) ZFHD1-p65 and ZFHD1-VP16 Chimeric Proteins Activate Transcription of a Target Gene Linked to a Nucleotide Sequence Containing ZFHD1 Binding Sites.

HT1080 cells were grown in MEM (GIBCO BRL) supplemented with 10% Fetal Bovine Serum. Cells in 35 mm dishes were transiently transfected by lipofection as follows: 10, 50, 250 ng of ZFHD-activation domain fusion plasmids together with 1 μg of pZHWTx8SVSEAP plasmid DNA were added to a microfuge tube with pUC118 plasmid to a total of 2.5 μg DNA per tube . The DNA in each tube was then mixed with 20 μg lipofectamine in 200 μl OPTIMEM (GIBCO BRL). The DNA-lipofectamine mix was incubated at room temperature for 20 min. Another 800 μl of OPTIMEM was added to each tube, mixed and added to HT1080 cells previously washed with 1 ml DMEM (GIBCO BRL). The cells were incubated at 37%C for 5 hrs. At this time, the DNA4ipofectamine media was removed and the cells were refed with 2 ml MEM containing 10% Fetal Bovine Serum. After 24 hrs incubation at 37%C, 20 μl of media was removed and assayed for SEAP activity as described (Spencer et al., 1993).

Results

Both ZFHD1-VP16 and ZFHD1-p65 support transcriptional activation of a gene encoding SEAP linked to ZFHD1 binding sites. The results are shown in FIG. 4A of PCT/US95/16982.

(ii) FK1012-Dependent Transcriptional Activation with ZFHD1-FKBPx3 and FKBPx3-VP16 or FKBPx3-p65

293 cells were grown in D-MEM (Gibco BRL) supplemented with 10% Bovine Calf Serum. Cells in 35 mm dishes (2.5×10⁵ cells/dish) were transiently transfected with use of calcium phosphate precipitation (Ausubel et. al., 1994). Each dish received 375 ng pZHWTx8SVSEAP; 12ng pCGNNZFHD1-FKBPx3 and 25ng pCGNNFKBPx3-VP16 or pCGNNFKBPx3-p65. Following transfection, 2 ml fresh media was added and supplemented with FK1012 to the desired concentration. After a 24 hour incubation 100 ml aliquot of media was removed and assayed for SEAP activity as described (Spencer et. al., 1993).

Results

ZFHD1-FKBPx3 supports FK1012 dependent transcriptional activation in conjunction with FKBPx3-VP16 or FKBPx3-p65. Peak activation was observed at FK1012 concentration of 100 nM. See FIG. 4B of PCT/US95/16982.

(iii) Synthetic Dimerizer-Dependent Transcriptional Activation with ZFHD1-FKBPx3 and FKBPx3-VP16 or FKBPx3-p65

An analgous experiment was conducted using a wholly synthetic dimerizer, AP1510, in place of FK1012. Like FK1012, AP1510 is a divalent FKBP-binder and is capable of dimerizing chimeric proteins which contain FKBP domains. In this experiment, 293 cells were grown in DMEM supplemented with 10% Bovine Calf Serum. Cells in 10 cm dishes were transiently transfected by calcium phosphate precipitation (Natesan and Gilman, 1995, Mol. Cell Biol, 15, 5975-5982). Each plate received 1 μg of pZHWTx8SVSEAP reporter, 50 ng pCGNNZFHD1-FKBP3×3, 50 ng pCGNNF3p65 or pCGNNF3VP16. Following transfection, 2 ml fresh media was added and supplemented with AP1510 to the desired concentration. After 24 hrs, 100 μl of the media was assayed for SEAP activity as described (Spencer et al, 1993).

AP1510 may be prepared as described in PCT/US95/10559 (WO 96/06097).

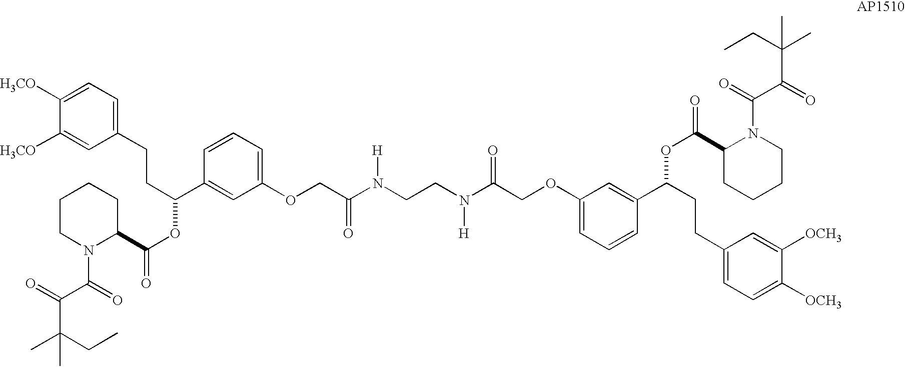

Results

ZFHD1-FKBPx3 supports synthetic dimerizer-dependent transcriptional activation in conjunction with FKBPx3VP16 or FKBPx3-p65. See FIG. 4C of PCT/US95/16982.

### Example 8

### Rapamycin-Dependent Transcriptional Activation with ZFHD1-FKBPx3 and FRAP-p65 in Whole Animals

Using the approach described in Example 7, constructs were prepared encoding the ZFHD1-FKBPx3 fusion protein, a second fusion protein containing the FKBP:rapaymcin binding (“FRB”) region of FRAP linked to the p6⁵ activation domain, and a reporter cassette containing a gene encoding human growth hormone linked to multiple ZFHD1 binding sites. The natural product, rapamycin, forms a ternary complex with FKBP12 and FRAP. Similarly, rapamycin is capable of binding to one or more of the FKBP domains and FRAP FRB domains of the fusion proteins. The three constructs were introduced into HT1080 cells which were then shown to support rapamycin-dependent expression of the hGH gene in cell culture, analogously to the experiments described in Example 7.

2×10⁶ cells from the transfected RT1080 culture were administered to nu/nu mice by intramuscular injection. Following cell implantation, rapamycin was administered i.v. over a range of doses (from 10-10,000 μg/kg). Serum samples were collected from the mice 17 hours after rapamycin administration. Control groups consisted of mice that received no cells but 1.0 mg/kg rapamycin (i.v.) as well as mice that received the cells but no rapamycin.

Dose-responsive expression of hGH was observed (as circulating hGH) over the range of rapamycin doses administered. Neither control group produced measurable hGH. The limit of detection of the hGH assay is 0.0125 ng/ml. See FIG. 5 of PCT/US95/16982.

These data show functional DNA binding of ZFHD1-FKBP(x3) to a ZFHD1 binding site in the context of dimerization with another fusion protein in whole animals. These data demonstrate that in vivo administration of a dimerizing agent can regulate gene expression in whole animals of secreted gene products from cells containing the fusion proteins and a responsive target gene cassette. It has also been found that a bolus hGH administration, either i.p. or i.v., results in rapid hGH clearance with a half-life of less than 2 minutes and undetectable levels by 30 minutes. Therefore, the observed hGH secretion in this example appears to be a sustained phenomenon.

### Example 9

### FRAP FRB Constructs

This Example provides further background and information relevant to constructs encoding chimeric proteins containing an FRB domain derived from FRAP for use in the practice of this invention. The VP16-FRB construct described below is analogous to the p65-FRB construct used Example 8.

Rapamycin is a natural product which binds to a FK506-binding protein, FKBP, to form a rapamycin:FKBP complex. That complex binds to the protein FRAP to form a ternary, [FKBP:rapamycin]:[FRAP], complex. The rapamycin-dependent association of FKBP12 and a 289 kDa mammalian protein termed FRAP, RAFT1 or RAPT1 and its yeast homologs DRR and TOR (hereafter refered to as “FRAP”) have been described by several research groups. See e.g. Brown et al, 1994, Nature 369:756-758, Sabatini et al, 1994, Cell 78:35-43, Chiu et al, 1994, Proc. Natl. Acad. Sci. USA 91:12574-12578, Chen et al, 1994, Biochem. Biophys. Res. Comm. 203:1-7, Kunz et al, 1993 Cell 73:585-596, Cafferkey et al, 1993 Mol. Cell. Biol. 13:6012-6023. Chiu et al, supra, and Stan et al, 1994, J. Biol. Chem. 269:32027-32030 describe the rapamycin-dependent binding of FKBP12 to smaller subunits of FRAP.

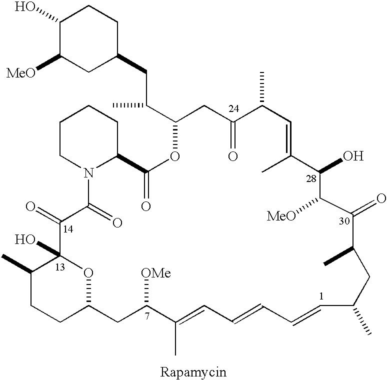

Construct Encoding FRAP domain(s)-VP16 Transcriptional Activation Domain(s)-Epitope Tag.

The starting point for assembling this construct was the eukaryotic expression vector pBJ5/NFlE, described in PCT/US94/01617. pBJ5 is a derivative of pCDL-SR (MCB 8,466-72) in which a polylinker containing 5′ SacI and 3′ EcoRI sites has been inserted between the 16S splice site and the poly A site. To construct pBJ5/NFlE a cassette was cloned into this polylinker that contained a Kozak sequence and start site, the coding sequence of the SV40 T antigen nudear localization sequence (NLS), a single FKBP domain, and an epitope tag from the H. influenza haemagglutinin protein (HA), flanked by restriction sites as shown below:

```
          Kozak SV40 NLS FKBP(5′)
                      M E D P K K K R K V L E G V Q V E ...
CCGCG\overline{GCCAC}CATGCTCGACCCTAAGAAGAAGAGAAAGGTACTCGAGGGCGTGCAGGTGGAG...
SacII (X/S) XhoI
      FKBP(3′) HA(flu)tag
... L L K\overline{ L E }V D Y P Y \overline{D V P D Y A } E D End
...CTTCTAAAACTGGAAGTCGACTATCCGTACGACGTACCAGACTACGCACTCGACTAAGAATTC
                                    SalI (X/S) EcoRI
```

where (X/S) denotes the result of a ligation event between the compatible products of digestion by XhoI and SalI, to produce a sequence that is cleavable by neither enzyme. Thus the XhoI and SalI sites that flank the FKBP coding sequence are unique.

The series of constructs encoding FRAP-VP16 fusions is assembled from pBJ5/NF1E in two steps: (i) the XhoI-SalI restriction fragment encoding FKBP is excised and replaced with fragments encompassing all or part of the coding sequence of human FRAP, obtained by PCR amplification, generating construct NR1E and relatives (where R denotes FRAP or a portion thereof; (ii) the coding sequence of the VP16 activation domain is cloned into the unique SalI site of these vectors to yield construct NR1V1E and relatives. At each stage additional manipulations are performed to generate constructs encoding multimers of the FRAP-derived and/or VP16 domains.

(i) Portions of human FRAP that include the region required for FRAP binding are amplified by PCR using a 5′ primer that contains a XhoI site and a 3′ primer that contains a SalI site. The amplified region can encode full-length FRAP (primers 1 and 4: fragment a); residues 2012 through 2144 (a 133 amino acid region that retains the ability to bind FKBP-rapamycin; see Chiu et al. (1994) Proc. Natl. Acad. Sci. USA 91: 12574-12578)(primers 2 and 5: fragment b); or residues 2025 through 2114 (a 90 amino acid region that also retains this ability; see Chen et al. (1995) Proc. Natl. Acad. Sci. USA 92: 49474951)(primers 3 and 6: fragment c). The DNA is amplified from human cDNA or a plasmid containing the FRAP gene by standard methods, and the PCR product is isolated and digested with SalI and XhoI. Plasmid pBJ5/NFlE is digested with SalI and XhoI and the cut vector purified. The digested PCR products are ligated into the cut vector to produce the constructs NRa1E, NRb1E and NRc1E, where Ra, Rb and Rc denote the full-length or partial FRAP fragments as indicated above. The constructs are verified by DNA sequencing.

Multimers of the FRAP domains are obtained by isolating the Ra, Rb or Rc sequences from the NRa1E, NRb1E and NRc1E vectors as XhoI/SalI fragments and then ligating these fragments back into the parental construct linearized with XhoI. Constructs containing two, three or more copies of the FRAP domain (designated NRa2E, NRa3E, NRb2E, NRb3E etc) are identified by restriction or PCR analysis and verified by DNA sequencing.

5′ ends of amplified products:

```
FRAP fragment a (full-length: primer 1)
                  L E L G T G P A A
5′ CGAGTCTCGAGCTTGGAACCGGACCTGCCGCC
                XhoI
FRAP fragment b (residues 2012-2144: primer 2)
                          L E V S E E L I R
5′ CGAGTCTCGAGGTGAGCGAGGAGCTGATCCGA
                XhoI
FRAP fragment c (residues 2025-2114: primer 3)
                  L E E M W H E G L
5′ CGAGTCTCGAGGAGATGTGGCATGAAGGCCTG
                XhoI
```

3′ ends of amplified products:

```
FRAP fragment a (full-length: primer 4)
        I G W C P F W V D
5′ ATTGGCTGGTGCCCTTTCTGGGTCGACCGAGT
3′ TAACCGACCACGGGAAAGACCCAGCTGGCTCA
                                                    SalI
FRAP fragment b (residues 2012-2144: primer 5)
        L A V P G T Y V D
5′ TTGGCTGTGCCAGGAACATATGTCGACCGAGT
3′ AACCGACACGGTCCTTGTATACAGCTGGCTCA
                                                    SalI
FRAP fragment c (residues 2012-2144: primer 6)
        F R R I S K Q V D
5′ TTCCGACGAATCTCAAAGCAGGTCGACCGAGT
3′ AAGGCTGCTTAGAGTTTCGTCCAGCTGGCTCA
                                                    SalI
```

(ii) The VP16 transcription activation domain (amino acids 413-490) is amplified by PCR using a 5′ primer (primer 7) containing a XhoI site and a 3′ primer (primer 8) containing a SalI site. The PCR product is isolated, digested with SalI and XhoI, and ligated into plasmid pBJ5/NF1E digested with SalI and XhoI to generate the intermediate NV1E. The construct is verified by restriction or PCR analysis and DNA sequencing. Multimerized VP16 domains are created by isolating the single VP16 sequence as a XhoI-SalI fragment from NV1E, and then ligating this fragment back into NV1E that is linearized with XhoI. This process generates constructs NV2E, NV3E and NV4E etc which can be identified by restriction or PCR analysis and verified by DNA sequencing.

5′ end of PCR Product:

```
                              413
                  L E A P P T D V
5′ CGACACTCGAGGCCCCCCCGACCGATGTC
                XhoI
```

3′ end of PCR Product:

```
                              490
        D E Y G G V D
5′ GACGAGTACGGTGGGGTCGACTGTCG
3′ CTGCTCATGCCACCCCAGCTGACAGC
                                      SalI
```

The final constructs encoding fusions of portions of FRAP with VP16 are created by transferring the VP16 sequences into the series of FRAP-encoding vectors described in (i). XhoI-SalI fragments encoding the 1, 2, 3 and 4 copies of the VP16 activation domains are generated by digestion of NV1E, NV2E, NV3E and NV4E. These fragments are then ligated into vectors NRa1E, NRb1E and NRc1E linearized with SalI, generating NRa1V1E, NRb1V1E, NRc1V1E, NRa1V2E, NRb1V2E, etc. Similarly, vectors encoding multiple copies of the FRAP domains are obtained by ligation of the same fragments into vectors NRa2E, NRa3E, NRb2E, NRb3E etc. All of these vectors are identified by restriction or PCR analysis and verified by DNA sequencing. Thus the final series of vectors encodes (from the N to the C terminus) a nuclear localization sequence, one or more FRAP-derived domains fused N-terminally to one or more VP16 transcription activation domains (contained on a single XhoI-SalI fragment), and an epitope tag.

Oligonucleotides:

| | |
|---|---|
| 1 | 5′ CGAGTCTCGAGCTTGGAACCGGACCTGCCGCC |
| 2 | 5′ CGAGTCTCGAGGTGAGCGAGGAGCTGATCCGA |
| 3 | 5′ CGAGTCTCGAGGAGATGTGGCATGAAGGCCTG |
| 4 | 5′ ACTCGGTCGACCCAGAAAGGGCACCAGCCAAT |
| 5 | 5′ ACTCGGTCGACATATGTTCCTGGCACAGCCAA |
| 6 | 5′ ACTCGGTCGACCTGCTTTGAGATTCGTCGGAA |
| 7 | 5′ CGACACTCGAGGCCCCCCCGACCGATGTC |
| 8 | 5′ CGACAGTCGACCCCACCGTACTCGTC |

Sequence of representative final construct (NRc1V1E):

```
        Kozak SV40 NLS FRAP(2025-2114)
                      M E D P K K K R K V L E E M W H E ...
CCGCGGCCACCATGCTCGACCCTAAGAAGAAGAGAAAGGTACTCGAGGAGATGTGGCATGAA...
SacII (X/S) XhoI
            FRAP(2025-2114) VP16(413-490)  . . . VP16(413-490)
... R I S K Q V D A P P T D D E Y G G V D
... CGAATCTCAAAGCAGGTCGAGGCCCCCCCGACCGAT...GACGAGTACGGTGGGGTCGAC
                                          (S/X) SalI
HA(flu)tag
  Y P Y D V P D Y A E D End
TATCCGTACGACGTACCAGACTACGCACTCGACTAAGAATTC
                                                          (X/S) EcoRI
```

For additional details and guidance on materials and methods for regulatable transcription based on rapamycin or analogs thereof, see PCT/US96/09948.

### Example 10

### Cloning IFN-Gamma, IL-10, Endothelial NO Synthase and IL-12 for Regulated Expression Under Dimerizer Control

Target gene casettes for the regulated expression of IFN-gamma IL-10, endothelial NO synthase or IL-12 may be prepared by analogy to constructs such as pZHWTx8SVSEAP and the corresponding target gene cassette used for regulated expression of hGH (again, see PCT/US96/09948). While the choice of DNA-binding domain and corresponding recognition sequence is left to the practitioner, the following experiments illustrate the use of the the ZFHD1 composite DNA-binding domain and its recognition sequence.

Constructs in which the expression of human IFN-gamma, IL-10, or endothelial NO synthase is placed under the control of a transcription factor utilizing the chimeric DNA binding domain ZFHD1 (Pomerantz et al., 1995) are prepared from the vector pZHWTxl2-CMV-SEAP (PCT/US96/09948), in which expression of the SEAP reporter gene is driven by a basal promoter from the immediate early gene of human cytomegalovirus (Boshart et al., 1985) downstream from 12 tandem copies of a ZFHD1 binding site. The complete gene sequence for IL-10 or IFN-gamma is amplified by PCR from a human genomic DNA library, or from an appropriate purified clone, with primers designed using the known gene sequences (Genbank accession numbers U16720 for IL-10, J00219 M37265 V00536 for IFN-gamma) as a guide. Example primers are 1 and 2 (for IL-10) or 3 and 4 (for IFN-gamma). The fragments are purified and digested with HindIII and EcoRI, sites appended by the PCR primers. pZHWTx12-CMV-SEAP is digested with HindIII and EcoRI to remove the SEAP coding sequence, and the digested PCR products are ligated in. Clones are confirmed by restriction digestion, PCR screening and/or DNA sequence analysis.

In some embodiments it will be preferable to express IL-10, IFN-gamma and/or NO synthase from constructs containing a cDNA rather than the complete gene including introns: for example, those cases in which the genes are to be introduced using a retroviral vector, or those cases in which the complete gene is especially large or the DNA coding capacity of the delivery vector limited. In these cases, cDNAs encoding the genes are amplified from mRNA from an appropriate human tissue source by RT-PCT using primers designed using the known mRNA sequences (Genbank accession numbers M57627 for IL-10, M29383 for IFN-gamma, and M95296 for NO synthase) as a guide. Example primers are 5 and 6 (for IL-10), 7 and 8 (for IFN-gamma), or, 9 and 10 (for NO synthase). These fragments are cloned as described above.

Human IL-12 is a heterodimer of 35 kDa alpha and 40 kDa beta subunits encoded by separate genes. Therefore, to obtain expression of IL-12 under dimerizer control, expression of both genes must be driven by regulated promoters. This may be achieved using separate promoters, a single bidirectional promoter (Baron et al. 1995 Nucl. Acids. Res. 23:3605-3606), or by placing both genes under the control of a single promoter to produce a dicistronic transcript utilizing an internal ribosome entry sequence (IRES) from EMCV, as described by Zitvogel et al (1994) Human Gene Therapy 5, 1493-1506 For example, the cDNAs for the alpha and beta subunits are amplified from mRNA from an appropriate human tissue source by RT-PCT using primers designed using the known mRNA sequences (Genbank accession numbers M65271 M38443 for alpha, M65272 M38443 M38444 for beta) as a guide. Example primers are 11 and 12 (for alpha) and 13 and 14 (for beta). The fragment produced by PCR with 11 and 12 is cloned as described above (HindIII-EcoRI). Then, the fragment produced by PCR with 13 and 14 is cloned as a EcoRI-ClaI fragment (downstream of the first product). Finally, a fragment containing the EMCV IRES (obtained as described in PCT/US96/09948) is blunt-cloned into the opened EcoRI site of the two-gene construct.

Primer Sequences:

| | |
|---|---|
| 1 | GCATCAAGCTTCACAAGACAGACTTGCAAAAGAAGG |
| 2 | CCATAGAATTCGTCTATAGAGTCGCCACCCTGATGTC |
| 3 | GCATCAAGCTTTTTGGCTTAATTCTCTCGGAAACG |
| 4 | CCATAGAATTCAGATTTAAAATTCAAATATTGCAGGCAGGA |
| 5 | GCATCAAGCTTATGCACAGCTCAGCACTGCTCTGTTG |
| 6 | CCATAGAATTCTCAGAAACGTATCTTCATTGTCATGT |
| 7 | GCATCAAGCTTATGAAATATACAAGTTATATCTT |
| 8 | CCATAGAATTCTTACTGGGATGCTCTTCGAGCTCGAA |
| 9 | GCATCAAGCTTCAGAGTGGACGCACAGTAACATGGG |
| 10 | CCATAGAATTCAAGGGAAAGCCAGGCGGCTCTCAGG |
| 11 | GCATCAAGCTTATGTGTCCAGCGCGCAGCCTCCTCC |
| 12 | CCATAGAATTCTTAGGAAGCATTCAGATAGCTCGTC |
| 13 | GCATCGAATTCATGTGTCACCAGCAGTTGGTCATC |
| 14 | CCATAATCGATCTAACTGCAGGGCACAGATGCCCAT |

Restriction sites used for cloning PCR products are underlined.

### Example 11

### Additional Experimental Details

The following experimental details are provided as further guidance to the practitioner. Information and approaches for subcloning, assembly of constructs and vectors and various components may be useful in the design and construction of transgenes for use in the practice of this invention.

### A. Direct Activation of Transcription

Transcription Factor Plasmid:

pCEN-F3p65/Z1F3/neo

Transcription factor fusion proteins and the neo gene are expressed from the mammalian expression vector pCEN, a derivative of pCGNN (7,8). Inserts cloned into pCEN as XbaI-BamHI fragments are transcribed under control of the human CMV enhancer/promoter (C) and are expressed with an amino-terminal epitope tag (E, a 16 amino acid portion of the influenza hemagglutinin [HA] gene) and nuclear localization sequence (N) from the SV40 large T antigen. pCEN-F3p65/Z1F3/neo produces a tricistronic transcript encoding the activation domain fusion 3xFKBP-p65 (F3p65), the DNA binding domain fusion ZFHD1-3xFKBP (Z1F3), and the neo gene, each separated by an internal ribosome entry sequence (IRES) from the encephalomyocarditis virus (see below). For human gene therapy applications, epitope tags are preferably omitted.

Target Plasmids:

LH-Z12-I-PL

This plasmid/retroviral vector contains long terminal repeats (LTRs) from the Moloney murine leukemia virus, one of which drives expression of the hygromycin resistance gene (see (3, 9)). Downstream of the hygromycin gene are 12 ZFD1 binding sites, a minimal human interleukin-2 (IL2) gene promoter and a polylinker. Insertion of the gene of interest into the polylinker puts its expression under control of the dimerizer-regulated transcription factors. Despite the presence of enhancers within the LTRs, in cell lines tested, this vector has low basal expression (3). Note that this vector can be used directly as a plasmid for transient transfections and for generating stable cell lines or it can be used to make retrovirus (see below). It is sufficient—and preferable when the vector is used to generate retrovirus—to insert only the coding sequence of the gene to be regulated, without the poly(A) signal or introns.

Alternatively, the ZFHD1-IL2 control region can be removed from this vector (using 5′ MluI or NheI sites and 3′ HindIII, PstI, EcoRI, SpeI, BglII or ClaI sites) and inserted upstream of the gene of interest. This may be preferred when it is important to maintain the genomic structure of the gene.

LH-Z12-I-S

This control vector contains the secreted alkaline phosphatase gene (a HindIII-ClaI fragment from pSEAP promoter vector, Clontech) inserted into LH-Z12-I-PL.

General Information

Procedure for Making Stable Cell Lines

I. Stably integrate the regulated transcription factors

A. Transfect cells with pCEN-F3p651Z1F3/neo

Linearization with SfiI enhances the efficiency of integration.

B. Select G418-resistant clones

>90% of G418-resistant clones should express the transcription factors.

C. Screen by transient transfection with LH-Z12-I-S (or another easily assayed reporter plasmid) for clones with low background and high dimerizer-dependent induction

The absolute level of inducibility of the reporter gene corrpression of the 3xFKBP-p65 activation domain fusion protein (˜68 kDa). Therefore, if desired, clones may first be screened by western using anti-HA antibodies (see below). Clones expressing the highest levels of 3xFKBP-p65 should be selected for further analysis by transfection.

II. Stably integrate the target plasmid

A. Transfect with plasmid vector or infect with retroviral vector containing the target gene under control of 12 ZFHD1-binding sites and a minimal IL2 promoter

If the LH-Z12-I-based plasmid vector is used, linearization with NotI or FspI will enhance the efficiency of integration.

B. Select hygromycin-resistant clones

C. Screen for clones with low background and high dimerizer-dependent induction of reporter gene expression

Transient Transfection Protocol

To screen clones it is convenient to transiently transfect cells in a 96-well format. Lipofectamine is used to introduce 50 ng total DNA/96-well under conditions recommended by the manufacturer (Gibco/BRL). If introducing both the transcription factor and target gene plasmids transiently, use 20 ng of each plasmid and 10 ng of carrier DNA (it may be necessary to optimize plasmid ratios and transfection conditions for each cell type). If only introducing one plasmid, bring to 50 ng with carrier DNA.

Following transfection, add medium +/−100 nM AP1510 dimerizer (or try a range of concentrations).

After overnight incubation (or longer), assay for target gene expression.

SEAP Assay Protocol

Secreted alkaline phosphatase activity can be easily measured from the supernatant of appropriately transfected cells using fluorescence-(see (10)), or chemiluminescence-(Tropix, Bedford, Mass.) based assays. Samples to be tested should first be incubated at 65° C. for 1 hour to inactivate endogenous alkaline phosphatase activity.

Western Protocol

The HA-epitope tagged transcription factors can be detected using commercially available anti-HA antibodies, including those from Babco (Richmond, CA; Cat. No. MMS101R-500). While the 3xFKBP-p65 activation domain fusion protein (˜68 kDa) should be easily detected, the DNA binding domain fusion, ZFHD1-3xFKBP (˜58 kDa), is expressed at lower levels and may not be visible. References for making retrovirus Helper-free retroviruses containing the target gene can be generated using the appropriate packaging vectors and cell lines as described elsewhere (9, 11).

Internal Ribosome Entry Sequence (IRES)

A tricistronic transcript expressing the transcription factor halves and the neo gene was created by inserting the ZFHD1-3xFKBP and neo genes downstream of the IRES from EMCV. To do this, 3 nucleotides, ACC, were added immediately 5′ to the 11th ATG of the EMCV IRES to create a Kozak consensus sequence and an NcoI site that encompasses the ATG. The ZFHD1-3xFKBP and neo genes were engineered to contain NcoI or compatible sites encompassing their ATGs which were then used to fuse the genes to the 11th ATG of the IRES. In the case of ZFHD1-3xFKBP, the amount of protein produced when it is the second cistron is only 10-20% of that produced when it is the first cistron. However, the relatively low level of expression of the DNA binding domain is still sufficient to direct high levels of induction of the target gene. Similarly, expression of the neo gene from the IRES is sufficient to confer resistance to G418.

Subcloning of the Transcription Factors

To put the expression of the transcription factors under control of an enhancer/promoter other than CMV, the coding region can be excised as a 4.94 kb EcoRI-BamHI fragment and subcloned. Note that this fragment must still be supplied with a poly(A) signal.

Alternatively, if the EcoRI site within the rabbit B-globin intron/poly A is mutagenized (see below), the CMV enhancer/promoter can be replaced as an EcoRI fragment.

### A. Indirect Activation of Transcription

(1) General Description

The reagents described here can be used to induce protein dimerization. To do this, the protein(s) of interest is fused to one or more copies of human FKBP12, which can be dimerized by AP1510. AP1510 can be used, for example, to homodimerize a receptor in order to mimic authentic ligand-induced dimerization or to alter the intracellular localization of a protein by recruiting it to another protein anchored at a different location in the cell. Regulated dimerization of a number of proteins using related dimerizers has been described (1, 10, 13-16).

The two plasmids included in this kit, pCFlE and pCMF2E, provide an assortment of components that can be easily manipulated to generate protein fusions whose activity and localization can be controlled by dimerizer.

(2) FKBP Expression Plasmids:

pCFIE

Inserts cloned into pCF1E as XbaI-SpeI fragments are transcribed under control of the human CMV enhancer promoter (C) and are expressed with a carboxy-terminal epitope tag (E, a 9 amino acid portion of the influenza hemagglutinin [HA] gene). The XbaI-SpeI insert in pCFlE contains a single copy of FKBP12 (F1). The amino terminus of this fusion protein (upstream of the XbaI site) consists only of a methionine and an alanine. Thus, the localization of the fusion protein is determined by that which is fused to FKBP12, since FKBP12 alone will be localized predominantly to the cytoplasm.

pCMF2E

Inserts cloned into pCMF2E as XbaI-SpeI fragments are transcribed under control of the human CMV enhancer promoter (C) and are expressed with an amino-terminal myristoylation-targeting peptide (M) from the amino terminus of v-src and a carboxy-terminal epitope tag (E, a 9 amino acid portion of the influenza hemagglutinin [HA] gene). The myristoylation sequence directs the fusion protein to cellular membranes. The XbaI-SpeI insert in pCMF2E contains two tandem copies of FKBP12 (F2).

(3) General Information

Cloning Strategy

The basic strategy for creating protein fusions in this example is to amplify the coding sequence of interest so that it contains the six nucleotides specifying an XbaI site immediately 5′ to the first codon (beware not to create an overlapping Dam methylation sequence) and the six nucleotides specifying a SpeI site immediately 3′ to the last codon. Then, for example, to fuse the protein amino terminal to 2 FKBPs, clone the XbaI-SpeI fragment into the XbaI site of pCMF2E (XbaI and SpeI have compatible cohesive ends). If inserted in the proper orientation, the XbaI and SpeI sites, now flanking the new fusion protein, will be maintained, with the junction of the two peptides consisting of the two amino acids specified by the SpeI and XbaI sites that were fused. Or to fuse the XbaI-SpeI fragment carboxy-terminal to 2 FKBPs, insert it into the SpeI site of pCMF2E. In both cases, since the flanking XbaI and SpeI sites are maintained, additional fragments can still be fused at the amino- and carboxy-terminal ends.

This strategy can also be applied to create 3 tandem FKBPs. For example, the XbaI-SpeI fragment of pCF1E can be inserted into the SpeI site of pCMF2E (or vice versa).

If the sequence to be fused contains internal XbaI or SpeI sites, fusions can still be made either by using XbaI or SpeI at both ends, or by using NheI or AvrII which also generate ends that are compatible with XbaI and SpeI. Note, though, that in these cases the flanking XbaI and SpeI sites will not be regenerated.

The sequence between the SpeI and BamHI sites of both vectors encodes a carboxy-terminal HA epitope tag followed by a stop codon. Therefore, stop codons should not be included in the fused sequences.

Finally, XbaI-SpeI or XbaI-BamHI fragments can be cloned into either the pCM- or pC-vector backbones to create fusion proteins containing or lacking amino-terminal myristoylation-targeting peptides, respectively.

Targeting Fusions to the Nucleus

Replacement of the XbaI-SpeI insert in pCEN-F3p65/ZlF3/neo with an XbaI-SpeI fragment containing an FKBP fusion will generate a fusion protein containing an amino-terminal HA epitope tag and a nuclear localization signal from the SV40 large T antigen.

Production of Single Stranded DNA for Mutagenesis/Sequencing

pCEN vectors contain an M13 ori for rescue of the antisense strand. Oligonucleotides used for mutagenesis or sequencing should correspond to the sense stand of the vector.

### C. Dimerizer

General Description

AP1510 is a synthetic dimerizer that can be used to induce homodimerization of FKBP12-containing fusion proteins. It is effective for both gene regulation applications and for general protein dimerization. AP1510 has no immunosuppressive activity and is non-toxic to cells.

AP1510 is conceptually related to FK1012, the prototype homodimerizer described in early dimerizer papers (10). Both molecules are symmetical homodimers of FKBP12 binding molecules. FK1012 is a semi-synthetic dimer of the natural product FK506. Positioning of the linker in the calcineurin binding domain of FK506 abolishes immunosuppressive activity while leaving FKBP12 binding unaffected. AP1510 is a smaller, simpler and completely synthetic molecule, in which two copies of an analog of the FK506 FKBP binding domain are directly linked. AP1510 generally outperforms FK1012 in gene regulation and protein dimerization applications. In gene regulation applications, AP1510 activates transcription at lower concentrations and to a higher level than PK1012. In addition, AP1510 activates transcription efficiently in cells in which the transcription factor and reporter gene constructs are all stably integrated, whereas the activity of FK1012 is poor under these conditions. AP1510 has also been successfully used to dimerize a number of transmembrane receptors that are activated by oligomerization.

As AP1510 is a completely synthetic molecule, it readily supports modification and optimization for a given application. A variety of other synthetic dimerizing agents are disclosed in WO 96/06097 and WO 97/31898 for binding to FKBP-related domains.

General Information

Reconstituting AP1510

AP1510 (molecular weight 1190 Da) may be stored in lyophilized form. It should be reconstituted as a concentrated stock in an organic solvent. It is recommended that the lyophilized material be dissolved in absolute ethanol to make a 1 MM solution (eg. dissolve 250 mg AP1510 in 210 ml ethanol). After adding the appropriate volume of ice-cold ethanol, seal and vortex periodically over a period of a few minutes to dissolve the compound. Keep on ice during dissolution to minimize evaporation.

Storing and Handling AP1510

Once dissolved, the stock solution can be kept at −20° C. indefinitely, in a glass vial or an eppendorf tube. Further dilutions in ethanol can be similarly stored. At the bench, solutions in ethanol should always be kept on ice, and opened for as short a time as possible, to prevent evaporation and consequent changes in concentration.

Using AP1510

Working concentrations of AP1510 can be obtained by adding compound directly from ethanol stocks, or by diluting serially in culture medium just before use. In the latter case it is recommended that the highest concentration not exceed 5 uM, to ensure complete solubility in the (aqueous) medium. In either case, the final concentration of ethanol in the medium added to mammalian cells should be kept below 0.5% (a 200-fold dilution of a 100% ethanol solution) to prevent detrimental effects of the solvent on the cells.

Expected Results

In gene regulation applications, expression becomes detectable at an AP1510 concentration of approximately 10 nM, and peaks at approximately 100 nM. When SEAP or hGH reporter systems are used, expression can be easily detected after an overnight incubation with dimerizer. In our experience, the efficacy is generally similar for other dimerization applications. A range of concentrations from 1 nM to 1 uM will typically provide a good dose-response profile in both cases.

### REFERENCES FOR THIS EXAMPLE

1. Belshaw, P. J., Ho, S. N., Crabtree, G. R., and Schreiber, S. L. Controlling protein association and subcellular localization with a synthetic ligand that induces heterodimerization of proteins. Proc. Natl. Acad. Sci 93, 4604-4607(1996).

2. Ho, S. N., Biggar, S. R., Spencer, D. M., Schreiber, S. L., and Crabtree, G. R. Dimeric ligands define a role for transcription activation domains in reinitiation. Nature 382, 822-826 (1996).

3. Rivera, V. M., Clackson, T., Natesan, S., Pollock, R., Amara, J. F., Keenan, T., Magari, S. R., Phillips, T., Courage, N. L., Cerasoli Jr., F., Holt, D. A., and Gilman, M. A humanized system for pharmacologic control of gene expression. Nature Med. 2, 1028-1032 (1996).

4. Pomerantz, J. L., Sharp, P. A., and Pabo, C. O. Structure-based design of transcription factors. Science 267, 93-96 (1995).

5. Schmitz, M. L. and Baeurle, P. A. The p65 subunit is responsible for the strong transcription activating potential of NF-kB. EMBO J. 10, 3805-3817 (1991).

6. Standaert, R. F., Galat, A., Verdine, G. L., and Schreiber, S. L. Molecular cloning and overexpression of the human FK506-binding protein FKBP. Nature 346, 671-674 (1990).

7. Attar, R. M. and Gilman, M. Z. Expression cloning of a novel zinc-finger protein that binds to the c-fos serum response element. Mol. Cell. Biol. 12, 2432-2443 (1992).

8. Tanaka, M. and Herr, W. Differential transcriptional activation by October-1 and October-2: Interdependent activation domains induce Oct-2 phosphorylation. Cell 60, 375-386 (1990).

9. Morgenstern, J. P. and Land, H. Advanced marnmalian gene transfer: high titre retroviral vectors with muplitple drug selection markers and a complementary helper-free packaging cell line. Nucl. Acids Res. 18, 3587-3596 (1990).

10. Spencer, D. M., Wandless, T. J., Schreiber, S. L., and Crabtree, G. R. Controlling signal transduction with synthetic ligands. Science 262, 1019-1024 (1993).

11. Pear, W. S., Nolan, G. P., Scott, M. L., and Baltimore, D. Production of high-titer helper-free retroviruses by transient transfection. Proc. Natl. Acad. Sci. USA 90, 8392-8396 (1993).

12. Rivera, V. M. and Gilman, M. Unpublished observations.

13. Luo, Z., Tzivion, G., Belshaw, P., Vavvas, D., Marshall, M., and Avruch, J. Oligomerization activates c-Raf-1 through a Ras-dependent mechanism. Nature 383, 181-184 (1996).

14. Holsinger, L. J., Spencer, D. M., Austin, D. J., Schreiber, S. L., and Crabtree, G. R. Signal transduction in T lymphocytes using a conditional allele of Sos. Proc. Natl. Acad. Sci. USA 92, 9810-9814 (1995).

15. Spencer, D. M., Graef, I., Austin, D. J., Schreiber, S. L., and Crabtree, G. R. A general strategy for producing conditional alleles of Src-like tyrosine kinases. Proc. Natl. Acad. Sci. USA 92, 9805-9809 (1995).

16. Spencer, D. M., Belshaw, P. J., Chen, L., Ho, S. N., Randazzo, F., Crabtree, G. R., and Schreiber, S. L. Functional anaysis of Fas signaling in vivo using synthetic inducers of dimerization. Curr. Biol. 6, 839-847 (1996).

### Example 12

### Methodology for Obtaining Bronchoalveolar Lavage for Evaluation of Indicators of Airway Inflammation

Male 200-250 gram guinea pigs are obtained from Charles River Laboratories and housed in separate polycarbonate boxes with free access to food and water. Boxes are changed twice a week and fresh bedding added. Temperature and humidity are maintained at 70° C. and 50% respectively. No other routine husbandry methods are necessary.

Homocytotropic antibodies (i.e., IgE) are generated by active sensitization. For sensitization to ovalbumin (OA), guinea pigs are injected (i.p.) on three occasions (day 1, day 14, and day 28) with 10 ug OA precipitated with 10 mg Al(OH)₃ in saline (total volume 0.5 ml/injection). One week following the final i.p. injection, the guinea pigs are used.

DNA is introduced into the animals as described elsewhere to render at least a portion of the cells in the animals' airways susceptible to drug-mediated expression of one or more target proteins. If desired, DNA introduction may be conducted prior to sensitization of the animals. In the week that the animals are to be used guinea pigs are administered OA via aerosolization as follows. Guinea pigs are placed in a 29 Liter aerosolization chamber and OA (0.1%) is nebulized from a DeVilbiss ultrasonic nebulizer (60 minutes, 1.5 ml/min) into the chamber. Sensitized, ovalbumin-challenged guinea pigs are euthanized with an overdose of pentobarbital (i.p., 100 mg/kg) at one of eight time points, ranging from 30 minutes to 48 hours after the OA challenge. Control sensitized guinea pigs receive aerosolized saline and are euthanized at an optimal time defined by the sensitized, ovalbumin-challenged guinea pigs. Unsensitized and sensitized, non-challenged guinea pigs also serve as control and are euthanized with pentobarbital. As soon as the animals expire, a mid-line incision is placed in the neck, just above the trachea, and extending to the xyphoid process over the sternum. The sternum is then longitudinally bisected and retracted to expose the lungs to ambient pressure. The trachea are excised and bisected in cross-section. A 15 gauge luer adapter is inserted into the trachea and tied with suture. The lung is then lavaged with hank's balanced salt solution (HBSS) with a volume equivalent to the functional residual capacity of the animal (30 ml/kg). This is repeated two more times. All volumes are collected.

At various times before or after the OA administration sensitized, ovalbumin-challenged guinea pigs are exposed to various doses of compounds or vehicle by one of the following routes: aerosol (20 minute nebulization; rate of 0.1 ml/min while guinea pigs are in a 29L aerosolization chamber), i.p. (1.0 ml total volume); p.o. (lavage, 1.0 ml total volume) or i.v. (penile vein, 1.0 ml total volume). At an optimal time defined by preliminary experiments described above, the guinea pigs are euthanized and lavaged as described.

Lavage fluid is analyzed for cells and/or mediators which correlate with hyperreactivity (e.g. total leukocyte counts, leukocyte differentials and/or levels of various TH2-type cytokines) and/or levels of target gene expression or target gene product activity. Normal levels of the former correlate with the prevention or alleviation of airway inflammation.

### Example 13

### Method For Evaluating Pulmonary Function

Male 200-250 gram guinea pigs are obtained from Charles River Laboratories and housed in separate polycarbonate boxes with free access to food and water. Boxes are changed twice a week and fresh bedding added. Temperature and humidity are maintained at 70C and 50%, respectively. No other routine husbandry methods are necessary.

Homocytotropic antibodies (i.e., IgE) are generated by active sensitization. For sensitization to ovalbumin (OA), guinea pigs are injected (i.p.) on three occasions (day 1, day 14, and day 28) with 10 ug OA precipitated with 10 mg Al(OH)₃ in saline (total volume 0.5 ml/injection). One week following the final i.p. injection, the guinea pigs are used.

DNA is introduced into the animals as described elsewhere to render at least a portion of the cells in the animals' airways susceptible to drug-mediated expression of one or more target proteins. If desired, DNA introduction may be conducted prior to sensitization of the animals.

On the week following the last i.p. injection, sensitized guinea pigs are utilized. They are anesthetized with 45 mg/kg sodium pentobarbital. Once anesthetized, a mid-line incision is placed in the neck, just above the trachea, and extending to the clavicle. The trachea is excised, debrided, and bisected in cross-section. A 15 gauge luer adapter is inserted into the trachea and tied in place with suture. An incision is placed in the skin, over the rib cage on the left side. The guinea pig is then placed in a single chamber whole body plethysmograph. The luer adapter is connected to a ventilator and the animal receives 4.0 ml tidal breaths at a rate of 50 breaths per minute. The animal then receives 0.5 mg/kg (i.p.) succinyl chloride to prevent any spontaneous breathng movements. A small whole is placed (access via the incision on the left flank) in an intercostal space of the 9th to 12th ribs and a PE100 catheter is introduced such that tip lies in the thoracic cavity at the supra-diaphragmatic margin. Ventilatory air flow and transpulmonary pressure are measured simultaneously on a Modular Instruments Inc. data acquisition system.

Baseline Vt Vexp, f, RL and GL are measured in real time. After baseline values are assessed, either acute bronchoconstriction or airway hyperreactivity measurements are begun. For acute bronchoconstriction methodology, ovalbumin (0.1%, 2 to 10 breaths) is administered via nebulization into the ventilator and ultimately the animal. GL is measured continuously thereafter. For airway hyperreactivity methodology, ascending doubling concentrations of methacholine (MeCH) are aerosolized into the nasal chamber of the plethysmograph in order to decrease GL. After MeCH administration (2 breaths), nadir GL is measured. This provides an index of the severity of MeCH induced bronchoconstriction. The provocative MeCH concentrations that produce reductions in GL are determined and used as indices of airway reactivity. The anesthetized, paralyzed guinea pigs are sacrificed by removing them from the plethysmograph/ventilator.

Airway hyperreactivity is induced in conscious guinea pigs by exposure to aerosolized ovalbumin (0.1%, 60 min., 1.5 ml/min) generated by a DeVilbiss ultrasonic nebulizer in a large aerosolization chamber. The animals may or may not be protected against severe pulmonary anaphylactic responses with anti-histamines (10 mg/kg diphenhydramine, i.p.; one hour prior to the ovalbumin challenge). Lack of protection mirniics the clinical situation of acute bronchoconstriction. In cases where animals are not protected, aerosolized ovalbumin exposure is conducted cautiously and incrementally over the first 2 minutes until animals are well adjusted to their new ventilatory patterns that result from acute bronchoconstriction. Twenty-four hours following antigen challenge, guinea pigs are placed in the plethysmograph and airway reactivity measured.

Animals are exposed to either (a) aerosolized antigen and vehicle or (b) antigen and compounds. Guinea pigs are exposed to various doses of compounds or vehicle by one of the following routes at various times either before or after the antigen challenge: aerosol (20 minute nebulization; rate of 0.1 ml/min), i.p. (1.0 ml total volume); p.o. (gavage, 1.0 ml total volume) or i.v. (penile vein, 1.0 ml total volume).

Abreviations: Vt=tidal volume; Vexp=expiration volume; f=frequency; RL=resistance of the lung; GL=conductance of the lung.

Each of the patent documents and scientific papers identified herein is hereby incorporated by reference. Those documents serve to illustrate the state of the art in various aspects of this invention. Numerous modifications and variations of the present invention should be apparent to one of skill in the art. Such modifications and variations, including design choices in selecting a regulated transcription system, ligand-binding domain, ligand, DNA binding domain, activation domain, DNA formulation, viral vector or other DNA delivery means, manner and route of transgene administration, in vivo models of pulmonary inflammation and function, in vitro assays of lymphocyte function or anti-inflammatory activity, etc. are believed to be encompassed by the scope of the invention and of the appended claims.

What is claimed is:

1. A method for genetically engineering mammalian cells to render them capable of regulated expression of a target gene comprising a DNA sequence encoding a target protein selected from the group consisting of IFN-gamma, IL-10, IL-12 and NO synthase, which method comprises introducing into the cells a target DNA construct comprising a target gene linked to a transcription control sequence permitting ligand-dependent expression of the target gene.

2. A method of claim 1 in which the cells are further engineered by the introduction into the cells of at least one regulatory DNA construct encoding a chimeric protein comprising any two or more of a transcription activating domain, a receptor domain capable of binding to a ligand, and a DNA binding domain, said target DNA construct comprising a gene encoding a target protein linked to a DNA sequence recognized by said DNA binding domain, said expression of the target gene is capable of being actuated by the presence of said ligand.

3. The method of any of claim 1 or 2, wherein the cells are present in the airways of a mammal.

4. The method of claim 3, wherein the mammal is a human.

5. The method of claim 4, wherein the target gene encodes a peptide sequence found in a naturally occurring human IFN-gamma, IL-10, IL-12 or NO synthase protein.

6. The method of any of claim 1 or 2, wherein the ligand has a molecular weight of less than 3 kD.

7. The method of any of claim 1 or 2 wherein the DNA constructs are introduced into the cells in one or more viral vectors.

8. The method of claim 4 wherein an aerosol formulation comprising the DNA constructs is introduced into the mammal's airways.

9. The method of claim 5 wherein an aerosol formulation comprising the DNA constructs is introduced into the mammal's airways.

10. The method of claim 6 wherein an aerosol formulation comprising the DNA constructs is introduced into the mammal's airways.

11. The method of claim 7 wherein an aerosol formulation comprising the DNA constructs is introduced into the mammal's airways.

12. A method for activating the expression in genetically engineered cells of at least one target selected from the group consisting of IFN-gamma, IL-10, IL-12 and NO synthase, wherein the genetically engineered cells had been genetically engineered by the introduction of one or more nucleic acid constructs encoding polypeptide(s) which activate the expression of said target gene(s) in a ligand-dependent manner, the method comprising administering said ligand to said genetically engineered cells.
